(19) United States
(12) Patent Application Publication
(10) Pub. No.: US 2024/0087688 A1
(43) Pub. Date: Mar. 14, 2024

(54) ARTIFICIAL INTELLIGENCE-BASED MODELING OF MOLECULAR SYSTEMS GUIDED BY QUANTUM MECHANICAL DATA

(71) Applicant: InterX, Inc., Berkeley, CA (US)

(72) Inventors: Boris FAIN, Berkeley, CA (US); Leonid PEREYASLAVETS, Lafayette, CA (US); Ganesh KAMATH, El Sobrante, CA (US); Igor KURNIKOV, Pittsburgh, PA (US); Serzhan SAKIPOV, Bridgeville, PA (US)

(73) Assignee: InterX, Inc., Berkeley, CA (US)

(21) Appl. No.: 18/136,621

(22) Filed: Apr. 19, 2023

Related U.S. Application Data
(60) Provisional application No. 63/403,120, filed on Sep. 1, 2022.

(51) Int. Cl. G16C 10/00; G06N 3/08; G16B 15/30; G16C 20/70

(52) U.S. Cl. CPC G16C 10/00; G06N 3/08; G16B 15/30; G16C 20/70

(57) ABSTRACT

A method includes determining a first set of atomic interactions between a first set of atoms and a second set of atoms using a molecular interaction model (EPh), providing inputs to a set of neural network models (dENN) to determine a second set of atomic interactions between the first set of atoms and the second set of atoms and training the dENN by reducing an error between an interaction produced by a combination of the first set of atomic interactions and the second set of atomic interactions and an interaction between the first set of atoms and the second set of atoms computed using quantum mechanical methods. The method includes reducing an error between the interaction produced by the combination of the first set of atomic interactions and the second set of atomic interactions and the interaction between the first set of atoms and the second set of atoms.

### CROSS-REFERENCE TO RELATED APPLICATION

This application claims priority to and the benefit of U.S. Provisional Patent Application No. 63/403,120, filed Sep. 1, 2022, and titled “Artificial Intelligence-Based Knowledge Processing Systems,” which is incorporated herein by reference in its entirety.

### TECHNICAL FIELD

The present disclosure generally relates to computer-implemented methods and systems of determining material properties of molecular quantum mechanical systems using artificial intelligence methods. In particular, but not by way of limitation, some embodiments described herein relate to computer-implemented methods and systems of determining material properties of molecular quantum mechanical systems according to artificial intelligence methods including, for example, using knowledge processing systems, such as neural networks, where a reasoning technique is applied to a collection of facts and relationships (i.e., knowledge representation).

### BACKGROUND

Molecular simulation is used to predict experimental observables of molecular systems. For example, molecular simulations may include force field models of a molecular system that represent the energy of the system (i.e., the Hamiltonian) which can be used to determine properties of the molecular system. Artificial intelligence methods such as supervised, unsupervised, and reinforcement learning have been employed to classify and predict physical, chemical, and biological systems. However, using artificial intelligence (AI) techniques to devise interaction models or force field (FF) models for complex molecules has been challenging, especially for those strongly affected by electrostatic and polarization effects. Furthermore, applying AI techniques to large molecular systems has also proven to be difficult. Additionally, some known molecular force field models and simulation methods that do not use quantum mechanical description (e.g., analytical force field models) may either have limited coverage of molecular systems or be unable to determine properties with an acceptable level of chemical accuracy.

Accordingly, a need exists for a wide coverage force field model and a molecular simulation method based on artificial intelligence techniques that are transferable and extendable to previously un-benchmarked chemical species, have little and/or minimum reliance on experimental data, and predict experimentally observable properties of molecular systems within an acceptable degree of chemical accuracy.

### SUMMARY

In some embodiments, a method includes determining, at a processor, a first set of atomic interactions between a first set of atoms and a second set of atoms using a molecular interaction model (EPh). The EPh can include responses to an external electric field, an inductive accounting of many-body non-additive interactions, and a representation of electrostatic interactions and electrostatic response interactions. The method further includes providing a set of inputs to a set of neural network models (dENN) to determine a second set of atomic interactions between the first set of atoms and the second set of atoms. Each input from the set of inputs is a representation of at least one atom of the first set of atoms or the second set of atoms. The method includes training the dENN by reducing an error between (1) an interaction produced by a combination of the first set of atomic interactions and the second set of atomic interactions and (2) an interaction between the first set of atoms and the second set of atoms computed using quantum mechanical methods. The method further includes modifying the EPh by reducing an error between (1) the interaction produced by the combination of the first set of atomic interactions and the second set of atomic interactions and (2) the interaction between the first set of atoms and the second set of atoms computed using quantum mechanical methods. The method includes receiving a representation of a third set of atoms, inputting the representation of the third set of atoms into the EPh and the dENN to output a prediction associated with an energy or a state of the third set of atoms, and sending a signal to a user device to present the prediction.

### BRIEF DESCRIPTION OF THE DRAWINGS

FIG. 1 is a schematic diagram of a molecular analysis device including models for computing atomic interactions, according to some embodiments.

FIG. 2 is a schematic of an atomic environment for an atom defined by a cutoff radius, according to some embodiments.

FIG. 3 is a structure of a high-dimensional neural network (NN), based on atomic energy contributions, according to some embodiments.

FIG. 4 is a schematic of an atomic environment for a pair of atoms defined by a cutoff radius, according to some embodiments.

FIG. 5 is a structure of a high-dimensional neural network (NN), based on atomic energy contributions from atomic pairs, according to some embodiments.

FIG. 6 is a representation of an environment of a pair of atoms, according to some embodiments.

FIG. 7 is an example of a set of radial functions, according to some embodiments.

FIG. 8 is an example of an energy error for an interaction between two water molecules, the energy error obtained as a difference between energy prediction obtained using models for computing atomic interactions and energy predictions computed using quantum mechanical calculations, according to some embodiments.

FIG. 9 is an example of an energy error for an interaction between a water molecule and a negative Cl ion, the energy error obtained as a difference between energy prediction obtained using models for computing atomic interactions and energy predictions computed using quantum mechanical calculations, according to some embodiments.

FIG. 10 is an example of an energy error for an interaction between a water molecule and a positive Na ion, the energy error obtained as a difference between energy prediction obtained using models for computing atomic interactions and energy predictions computed using quantum mechanical calculations, according to some embodiments.

FIG. 11 is an example of an energy error for interaction of different molecules, the energy error obtained as a difference between the energy predictions obtained using models for computing atomic interactions and the energy predictions computed using quantum mechanical calculations, according to some embodiments.

FIG. 12A is a schematic of an interaction between a molecule of water and a molecule of methane, according to some embodiments.

FIG. 12B is a schematic of an interaction between two molecules, according to some embodiments.

FIG. 13 is a schematic representation of a system of molecules with intermolecular interactions described by an intermolecular hybrid model, according to some embodiments.

FIG. 14 is a method of predicting an energy or a state of a set of atoms, according to some embodiments.

FIG. 15 is method of predicting an energy or a state of a set of atoms, according to some embodiments.

FIG. 16 is an example schematic of an intermolecular hybrid model being a combination of molecular interaction model and a computer learning model, according to some embodiments.

### DETAILED DESCRIPTION

A force field is a method that can be used to estimate the forces between atoms within molecules and also between molecules. The force field refers to the functional form and parameter sets used to calculate the potential energy of a system of atoms or coarse-grained particles in molecular mechanics, molecular dynamics, or Monte Carlo simulations. The parameters for a chosen energy function can be derived or adjusted from experiments in physics and chemistry, calculations in quantum mechanics, or both.

The force field parameters and interaction rules in applications to molecular systems can define the energy landscape from which a number of results of interest can be derived. For example, the acting forces on particles can be derived as a gradient of the potential energy with respect to the particle coordinates. For another example, the resulting equations of motion can be propagated forward in time and time averages can predict experimental observables.

Prediction of experimentally observable ensemble and non-ensemble properties of a material system may be obtained from a molecular force field model of the material system. An ensemble property may be obtained via molecular simulation of the model, for example, molecular dynamics (MD), path integral molecular dynamics (PIMD), or Monte Carlo (MC). The way to determine the ensemble properties from the Hamiltonian at non-zero temperature can be, for example, via various thermodynamic sampling methods such as molecular dynamics (MD), path integral molecular dynamics (PIMD), or Monte Carlo (MC).

The molecular interaction models can be roughly separated into two categories: scope limited models that benchmark their interaction energies with Quantum Mechanics (QM) calculations, and wide coverage models that fit at least some of their parameters to selected sets of experimental data. Some known QM parametrized models can employ high functional complexity to reproduce QM energies. The functional complexity of these known QM-parameterized models, however, can make transferability and extending coverage to other chemical species and hetero-chemical ensembles difficult.

Some known wide-coverage molecular force field models derive some or all of the parameters by fitting to empirical observables. There are at least two drawbacks to this approach. First, even available experimental data (e.g., densities, heats of vaporization) can be insufficient to produce models that describe existing compounds precisely; and there can be molecules (for example, molecules that have not been synthesized) that require and/or use more precise description than available data from existing inference. Second, if an empirical model's prediction is erroneous, it can be difficult to decide which parameter(s) of the model to remove, add, correct or adjust.

In a wide coverage force field, a chemical group can be considered described when both its self-interactions and its interactions with other described group(s) are determined. A Force Field with wide coverage can, in some examples, fully describe the interactions between most and/or all chemical groups. In the case of a protein, because of the importance of biology, wide coverage force fields can include the interaction of amino acids and backbone, for example, both between themselves, water, and non-polar solvents. Furthermore, in some instances, the force field can describe the interactions between a significant number of major chemical groups, e.g., alkanes aromatics amides amines sulfides esters, in both homo (self) and hetero (between any distinct number of) interactions. More specifically, in some instances, the force field can describe the behavior of these groups in a variety of solvents: H2O, CHEX (cyclohexane), OCT (octanol), Benzene, etc.

Finally, the simulation results produced with the force-field can correctly describe the entropic behavior of major solvents—H2O, CHEX, OCT, benzene, in order to capture the entropic component of solvation. Moreover, the results can correctly describe the entropic behavior of arbitrary molecular systems.

Some known fixed charge force field models lack flexibilities in the functional form to fit the experimental results well and frequently the errors are uncontrollable. The parameters of these known fixed charge force field models that are determined by QM energies are not well defined, as these models do not have polarization, and are, therefore, unable to transfer the results obtained by QM (in the gas phase) into the liquid phase or the solid phase (condensed phase). Furthermore, when fitting to experimental results, these known models do not have clear guidance on which parameters should be corrected, and therefore errors also arise.

One of the goals of a molecular simulation is to accurately reproduce the true quantum mechanical potential energy with a tractable classical approximation. In general, analytical expressions may not represent the intermolecular quantum potential energy surface at close distances and in strong interaction regimes. Alternatively, a description based on a neural network may be used. However, in some cases, the more accurate neural network approximations may not capture crucial physics concepts (e.g., many-body contributions and application of electric fields).

For such cases, a hybrid intermolecular interaction model may be used. The intermolecular interaction model may include analytical description of the polarizable force-field combined with a short-range neural network correction for the total intermolecular interaction energy. The correction is sub-typed for distinct chemical species to match the underlying force-field, to segment and reduce the amount of quantum training data, and to increase accuracy and computational speed. Simulations of molecular systems using such interaction model may determine properties of complex molecular systems at a fraction of time of some known simulation models.

Example embodiments are illustrated in the drawings. It is to be understood that the embodiments are described only to facilitate better understanding of the embodiments, rather than to limit the scope of the embodiments in any manner.

### System

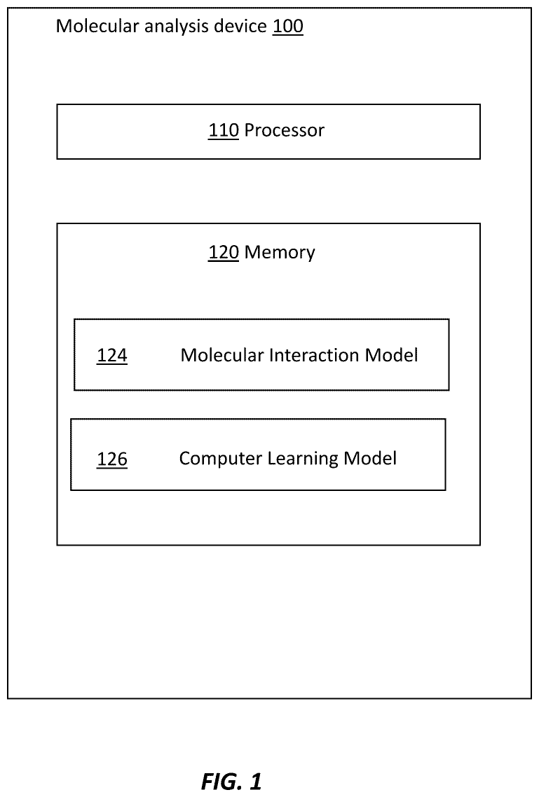

FIG. 1 illustrates an example schematic diagram illustrating the molecular analysis device (MAD) 100, according to some embodiments. The MAD 100 can be a compute device (or multiple compute devices) having a processor 110 and a memory 120 operatively coupled to the processor 110 (herein, the processor 110 can be used as a collective term for multiple processors, and memory 120 can be used as a collective term for multiple memory elements). In some instances, the MAD 100 can be any combination of hardware-based component (e.g., a field-programmable gate array (FPGA), an application specific integrated circuit (ASIC), a digital signal processor (DSP), a graphics processing unit (GPU)) and/or software-based component (computer code stored in memory 120 and/or executed at the processor 110) capable of performing one or more specific functions associated with that component. In some instances, the MAD 100 can be a server such as, for example, a web server, an application server, a proxy server, a telnet server, a file transfer protocol (FTP) server, a mail server, a list server, a collaboration server and/or the like. In some instances, the MAD 100 can be a personal computing device such as a desktop computer, a laptop computer, a personal digital assistant (PDA), a mobile telephone, a tablet personal computer (PC), and/or so forth.

The memory 120 can be, for example, a random-access memory (RAM) (e.g., a dynamic RAM, a static RAM), a flash memory, a removable memory, a hard drive, a database and/or so forth. In some implementations, the memory 120 can include (or store), for example, a database, process, application, virtual machine, and/or other software modules (stored and/or executed in hardware) and/or hardware modules configured to execute various molecular simulation process(es) as further described below. In such implementations, instructions for executing various molecular simulation process(es) and/or the associated methods can be stored within the memory 120 and executed at the processor 110.

The processor 110 can be, for example, a system of parallel processors, a system of GPUs, and the like, configured to make computations in parallel. For example, processor 110 may be a blade server having multiple processors such as, for example, multiple Intel processors (e.g., Xenon processors), AMD processors (e.g., Ryzen or EPYC processors), and the like. The processor 110 may use any suitable operating system (e.g., Linux, Window, Mac OS, Unix, and the like) to perform various processing functions. Such functions may include, for example, writing data into and reading data from the memory 120, and executing various instructions stored within the memory 120. The processor 110 can also be configured to execute and/or control, for example, the operations of other components of the MAD 100 (such as a network interface card, other peripheral processing components (not shown)).

The memory 120 is configured to store models for performing various molecular simulation process(es). In particular, the memory 120 is configured to store a molecular interaction model (MIM) 124 (herein and elsewhere, MIM is also referred to as EPh) and a computer learning model (CLM) 126 (herein and elsewhere, CLM is also referred to as neural network model or dENN). The MIM 124 and the CLM 126 can be executed by the processor 110. Accordingly, the MIM 124 and the CLM 126 can be software models stored in memory 120 and executed by processor 110. In other embodiments, the MIM 124 and CLM 126 can be hardware-based models.

The MIM 124 provides a physics-based model of a multidimensional potential-energy surface (PES) associated with atomic coordinates in the molecular system. Physical-based models include classical force fields for calculating the total energy of a molecular system. The total energy of the molecular system may be decomposed into low-dimensional bonding two-, three-, and four-body terms representing covalent bonds, bonding angles, and dihedral angles. In addition, electrostatic and van der Waals interactions given by Coulomb's law and the Lennard-Jones potential, may be used. For example, in some cases of physics-based models the total energy may include bond energy terms (e.g., bond energy may be a continuous function of interatomic distance and describes the energy associated with forming bonds between atoms), energy terms associated with three-body valence angle strain and four-body torsional angle strain, as well as electrostatic and van der Waals energy terms.

When computing low-dimensional bonding terms, the immediate environment may be taken into account. In various cases, the bonding properties are strongly influenced by the positions of the neighboring atoms, thus, additional information on the bonding properties of the atoms is included via the introduction of atom types classifying the atoms according to functional groups. In this way, total energy expressions with low-dimensional, approximately additive energy terms can be obtained. Further, reactive force fields (e.g., ReaxFF method) are used for describing the making and breaking of bonds. Some atomistic potentials in the field of materials science are based on the concept of the bond order to take into account the effect of the atomic environments on the bonding properties. In many problems of materials science involving systems such as metals and alloys a good description of very different atomic environments is important due to the importance of many-body contributions to the potential-energy.

In some cases, MIM 124 may include two-body non-bonded interactions including electrostatic, exchange-repulsion and dispersion terms. The electrostatic interaction arises from the attraction between atoms with opposite charges and the repulsion between atoms with like charges. This interaction is described by Coulomb's law. The exchange-repulsion interaction arises from the overlap of the electron clouds of two atoms. This overlap creates a repulsive force that is proportional to the overlap volume and the fourth power of the distance between the atoms. This interaction is typically described by a Lennard-Jones potential, which combines an attractive term that describes the van der Waals interaction between the atoms and a repulsive term that accounts for the exchange-repulsion interaction. The electrostatic and exchange-repulsion terms are multipolar, and their radial dependence may be exponential so that they are better able to describe charge penetration and delocalization. The dispersion interaction arises from the fluctuations in the electron density of an atom, which induce corresponding fluctuations in the electron density of neighboring atoms. This interaction results in an attractive force between the atoms whose leading term is proportional to the inverse sixth power of the distance between them. In molecular mechanics calculations, the dispersion interaction between two atoms is typically described by a function that depends on their polarizabilities and the distance between them. The dispersion term may be conventionally represented by a spherical, 2 term (C6 and C8), Tang-Toennies-damped interaction. Many-body effects can be modeled by anisotropic atomic polarizable dipoles interacting with the electrostatic term and with each other and iterated to self-consistent field (SCF) convergence on the non-bonded steps. The intermolecular parameters of MIM 124 may be determined by agreements with QM values of dimer and multimer energies, electrostatic potentials, and multipole moments of monomers. Further, MTM 124 may include bonded interactions that are fitted to monomer QM energies computed using df-MP2/aug-cc-pVTZ basis set.

In addition to physics-based model of a multidimensional potential-energy surface (PES), neural network-based (NN-based) models may be used to reproduce or encode atomic interactions. Some known NN-based models may be unable to incorporate effects which traditional physics has been built to represent such as, a response to an external electric field, the ability to extrapolate from 2 or 3 molecules to arbitrarily many, and a proper treatment and truncation of long-range interactions. Furthermore, the accuracy of the reproduction of intermolecular potentials by some known NN-based models has plateaued at −1 kcal/mol. Therefore, a combined hybrid intermolecular interaction model may be used. For example, as shown in FIG. 1, a computer learning model (CLM) 126 may be combined with the MIM 124 to result in the hybrid intermolecular interaction model.

The CLM 126 may be any suitable machine learning model for determining interactions between atoms of a molecular system. In some implementations, for example, the CLM 126 may be a system of neural networks (NN) incorporating description of a molecular neighborhood for determining atomic interactions. The CLM 126 may be trained using input data that includes reference data describing molecular structures and their corresponding energies. Further, CLM 126 may be validated using a suitable validation data set (e.g., the validation data set may include various molecular structures and their corresponding energies). In some cases, CLM 126 may be represented by a multilayer feed-forward (MLFF) NN. Such NNs can be useful for the construction of PESs due to their ability to represent arbitrary functions. Such NNs offer a number of advantages for the construction of PESs as energies of molecular structures can be fitted to a high degree of accuracy when compared to the underlying reference data. CLM 126 that uses NNs may be unbiased and generally applicable to various types of bonding and may not require system specific modifications.

In various embodiments, a demand for accuracy may be particularly important for protein-ligand interactions. For example, the energies of interest (interacting ligand and protein functional groups) are diverse and frequently quite strong. For another example, because the system is a liquid crystal and its phase space is constrained, the PES errors may not average out as in a liquid, and the model interactions can correctly encompass the distances and orientations. Consequently, the accuracy of each component of a useful prediction of the free energy of ligand binding can be significantly higher than the desired accuracy of the final answer (˜±0.5 kcal/mol). The components' agreement with Quantum Mechanics may be as narrow as the uncertainty of the underlying ab-initio calculations.

In various embodiments, the MIM 124 and CLM 126 are configured to be used together to describe an interaction of a group of atoms (e.g., atoms forming a molecular system) with predefined accuracy. In various embodiments, CLM 126 may be used for improving approximation of the MIM 124. For example, CLM 126 may be used for correcting a polarizable force field for (molecular) pair interactions at medium-short distances. In some cases, the correction encoding/computational mechanism represented by CLM 126 may be performed using neural networks (NNs). The combination of MIM 124 and CLM 126 is configured to provide energy predictions in agreement with quantum mechanical (QM) energies. Various aspects of a molecular interaction model similar to MIM 124 are described in U.S. patent application Ser. No. 17/967,451, filed on Oct. 17, 2022, and titled “System and Method for Determining Material Properties of Molecular Systems from an Ab-Initio Parameterized Force-Field,” which is incorporated by reference herein in its entirety. The interaction energy is formally assigned to (atomic) pair interactions (e.g., interactions between an atom A and an atom B) and takes into account the molecular neighborhoods of atom A and atom B therefore taking into account interactions with other atoms in the neighborhood of atoms A and B.

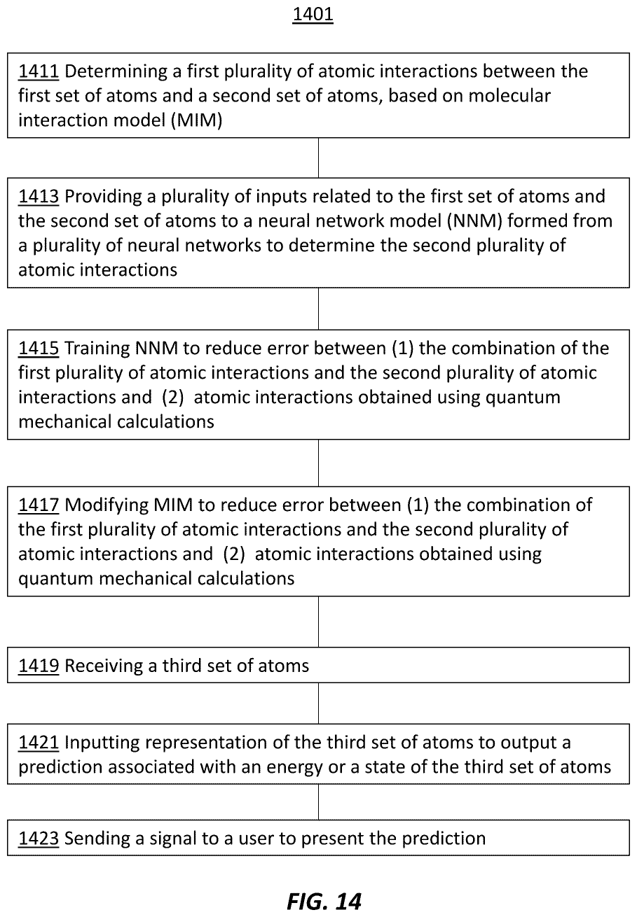

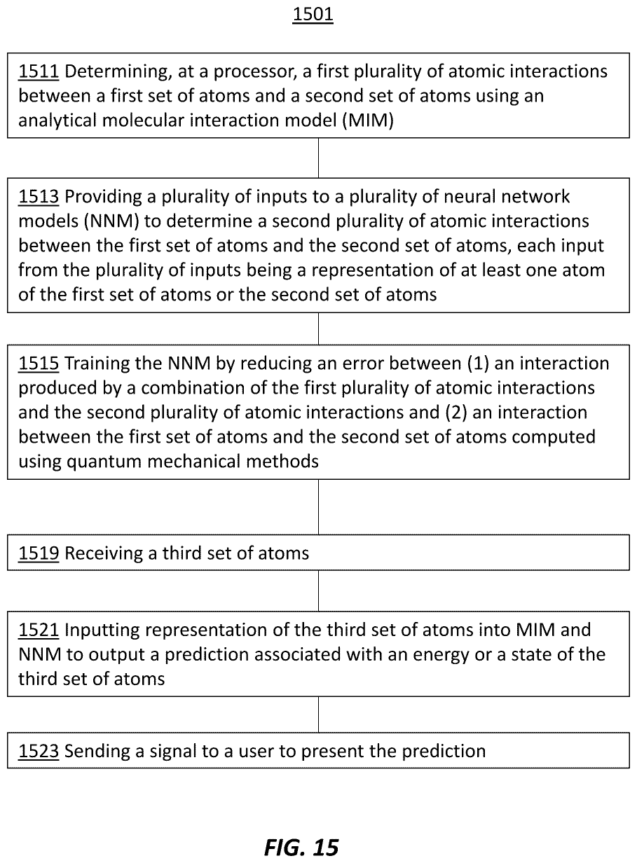

It should be noted MIM 124 and CLM 126 may be combined in a variety of ways to describe the interaction of a group of atoms with the predefined accuracy (e.g., accuracy which matches quantum mechanical predictions). For example, MTM 124 may be used for long range interactions and CLM 126 may be used for short range interactions. Corrections due to CLM 126 may be range dependent (e.g., corrections due to CLM 126 may be weighted by a function that is range dependent). Alternatively, MIM 124 may be used for describing interactions between a first set of atoms and CLM 126 may be used for describing interactions between a second set of atoms. In some cases, MIM 124 may be used for describing interactions between atoms of a molecular system (e.g., molecular system that includes two or more interacting molecules) and CLM 126 may be used to describe atomic interactions between a subset of atoms of that molecular system (e.g., atoms between the two molecules within the molecular system that are in proximity to each other). Further details of how MIM 124 and CLM 126 may be combined are discussed below in relation to methods shown in FIGS. 14 and 15.

In some implementations, the hybrid intermolecular interaction model (e.g., a model that combines MTM 124 and CLM 126) can address the following:
(a) At close intermolecular distances, the classical decompositions of quantum interactions (electrostatics, exchange-repulsion, dispersion, induction/charge transfer, etc.) may not be accurate. Alternatively, an accurate description of the above and of the total energy by analytical functional forms for distances and orientations may be prohibitively complex. The hybrid intermolecular interaction model allows for accurate description of molecular interactions without being prohibitively computationally expensive.
(b) The representations and decompositions of quantum interactions should be preserved as much as possible to retain the inferences made by physics, such as responses to electric fields, many-body effects, and long-range truncation. A physics-based description of polarization/induction is important as it allows models parameterized on small sets of molecules to be valid for condensed phase ensembles of arbitrary size. In some implementations, the hybrid intermolecular interaction model can maintain such representations and decompositions.
(c) In some implementations, the hybrid intermolecular interaction model is configured such that the remainder or the residual error of the description of the total 2-body QM energy by an analytical form is encoded in a trainable, highly flexible, and computationally economical object, such as a perceptron.
(d) In the regime of close molecular approach, in which the separability of pairwise atomic interactions from other interactions with other atoms in a molecular system begin to break down, neural networks can be used to determine the interaction energy. The proposed combination of an analytical polarizable Force Field with an intermolecular 2-body neural network short-range correction is a fast and accurate representation of the intermolecular potential energy surface.
(e) Because the residual error of the description of the total 2-body QM energy depends on the underlying analytical force-field, in some implementations, the hybrid intermolecular interaction model is configured such that the perceptron term may be (atom) typed as is the underlying force field. This can permit the pair-interaction specific neural network to be more economical because the neural network is not tasked with encoding all of chemical space. Moreover, this can allow the model to be incrementally enabled in distinct small slices of chemical interaction space with a relatively small training set of structures and energies.

Quantum mechanical calculations can become computationally expensive when molecules (or molecular systems) larger than a certain limited size (e.g., a few tens of atoms, a few hundreds of atoms, and/or the like) are considered. Therefore, in some implementations, analytical and computer models (e.g., MIM 124 and CLM 126) can be constructed using self-contained sub-pieces, which may be ‘stitched together’ along, for example, rotatable bonds, by fitting the ‘joining piece’ using monomer calculations or previous typification. For example, anthracene (3 joined aromatic rings) has the atom types identical to benzene except the 4 atoms at the joining sides, which are identical to naphthalene (2 joined aromatic benzene rings), thus, forces between atoms of anthracene may be calculated using MIM 124 and/or CLM 126 based on force calculations made for atoms of benzene.

When using MIM 124, it may be possible to identify many-body contributions from dimer calculations or very few multimer calculations by using an established physics theory and mechanism (polarization). In various cases, neural networks (NNs) may be too flexible and may be both provided and presented in training by the possible conformations to be encountered. If one needs to train many-body to a NN, the inputs can respond to and represent the multi-molecule interactions by including various permutations of such permutations, including, for example, various configurations of 4-mers.

Further, a proper description of electrostatics as well as polarization allows a system to parametrize the models and then conduct correct simulations, for example, in the presence of an electric field (e.g., in a battery). An example NN for interactions without such physics mechanisms can be trained using a large number of possible electric field configurations (e.g., electric fields both internal and externally applied). Thus, having CLM 126 for proper description of electrostatics may be computationally demanding. Therefore, in various implementations, MIM 124 may be used instead of CLM 126 for describing such interactions to reduce computation costs.

When describing a high-dimensional molecular system such as a molecular system having more than a few atoms (e.g., a molecular system with 5-10 atoms, with a tens of atoms, with hundreds of atoms or even with thousands of atoms), using a single neural network to describe interactions of the atoms in the molecular system may result in slow training times for such a network, thereby, CLM 126 may be formulated based on and/or include multiple NNs. Each NN can be configured to determine an energy contribution from a single atom to a total energy of the molecular system, and energy contributions from the atoms in the molecular system are then summed to obtain a total energy of the molecular system. Thus, to obtain a fitted high-dimensional PES, the total energy E of a molecular system can be decomposed into atomic energy contributions Eᵢ of each atom i, which are predicted by separated species-specific neural networks.

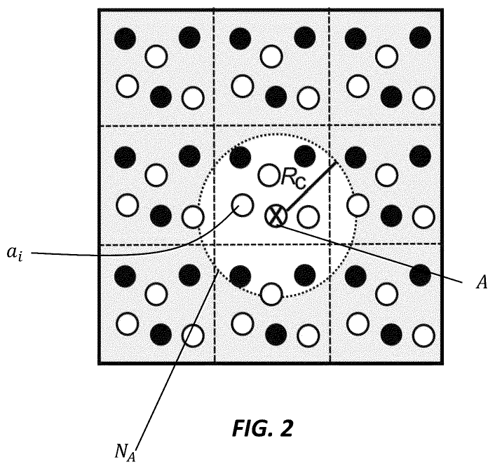

Further, when calculating interactions between a pair of atoms A and B, atoms aᵢ in a spherical neighborhood of atom A may be selected to describe the atomic environment functions G_{A} of atom A, and atoms bⱼ in a spherical neighborhood of atom B may be selected to describe the atomic environment functions G_{B} of atom B. The extent of neighborhoods of atoms A and B may be determined by a cutoff radius R_{C}, as schematically shown in FIG. 2 for a neighborhood N_{A} of atom A. As shown in FIG. 2, atoms aᵢ within neighborhood N_{A} may be counted towards energy calculations when calculating interaction between atoms A and B, while more distant atoms, outside neighborhood N_{A} defined by a sphere with the cutoff radius R_{C} may be excluded.

The cutoff radius R_{C} can be chosen sufficiently large such that accurate energy predictions can be obtained. For some systems R_{C} may be about 6 Å in order to reproduce energy predictions obtained using various methods based, for example, on quantum mechanical description such as methods based on, for example, density function theory (DFT), time-dependent density functional theory (TDDFT), Hartree-Fock theory, coupled cluster technique, DFT with projector-augmented wave (PAW) method, and/or Møller-Plesset perturbation theory (e.g., MP2). In various embodiments, the error for predicted energies may have a root mean squared error (RMSE) of only a few meV per atom. The specific geometric arrangement of the neighboring atoms is described by a set of atom-centered symmetry functions Gᵢ, which provide characteristic vectors of numbers for different local environments. The atom-centered symmetry functions Gᵢ may be similar to or the same as the atomic environment functions G_{A} and G_{B}. Herein, such functions are also referred to as atomic neighborhood descriptors.

As the decomposed atomic energies are not defined exactly, the training targets are the total energies, and this approach works with good accuracy for short-ranged interactions, while for long-ranged interactions as electrostatics and dispersion use special fitting and often are not considered. For many applications like liquid phase, biomolecular systems, molecular crystals, drugs, etc., the non-covalent interactions are of significance and can be taken into account. In some implementations, intermolecular interaction energy dEᵢₙₜ of two molecules can be calculated with a ‘supermolecular’ method as a difference between the total energies of a dimer, E_{AB}, and molecules/monomers, E_{A} and E_{B}. Thus, the intermolecular interaction energy is given as dEᵢₙₜ=E_{AB}−E_{A}−E_{B}. The dEᵢₙₜ calculated using ab initio quantum mechanical (QM) methods can be used for neural network training. In various cases, the proposed approach of the neural network potentials (NNP) may be used to describe non-covalent interactions.

In various embodiments, CLM 126 may take a variable input (e.g., the variable input may include symmetry function vectors Gᵢ describing atomic environments for atoms Aᵢ, and return a total energy of the molecular system. The total energy may be the atomization energy (i.e., the energy of forming a molecule from individual atoms). In various cases, CLM 126 may be used for correcting or augmenting the intermolecular energy of a molecular system. In an example embodiment, the molecular system may include a pair of interacting molecules.

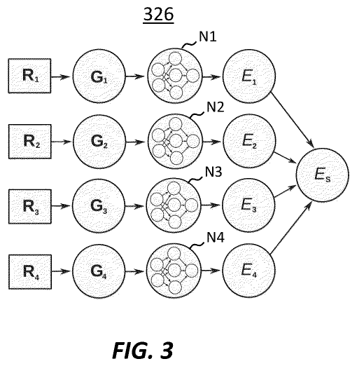

FIG. 3 shows an example CLM 326, which may be an example implementation of CLM 126. In an example embodiment, inputs R₁ . . . R₄ are coordinates of four atoms within the molecular system, G₁ . . . G₄ are atom-centered symmetry functions providing characteristic numbers for different local environments, and N₁ . . . N₄ are neural networks for determining respective energy contributions E₁ . . . E₄ for each one of the four atoms. The energy contributions E₁ . . . E₄ are then summed to obtain a total energy of the system E_{S}. In various embodiments, neural networks N₁ through N₄ may be specific to a type of atom for which the energy is computed. For example, a neural network for a carbon atom may not be the same as the neural network for a hydrogen atom. In some cases, neural networks N₁ through N₄ may be structurally the same (e.g., may include the same number of layers) but may have different weights. In some cases, the differentiation between different networks (e.g., the difference in weights of different networks) may be due to training. Further networks N₁ through N₄ may be different (e.g., such networks may have different weights) for an atom ‘type’ of an atom based on the atom's QM monomer properties and the atom's chemical environment in the molecule. The atom type can be a classification of an atom, which can depend not only on its atomic number but also based on its chemical connections to other atoms in the molecule. An example of such differentiating atom types can be an aromatic carbon and an aliphatic carbon.

In various embodiments, CLM 126 may be constructed and/or defined to be extensible. Thus, CLM 126 may be used for multiple subsets of atoms within a molecular system, and atomic interactions between various subsets of atoms can be combined to obtain an overall energy of the molecular system. In one implementation, the molecular system may be partitioned into pairs of atoms (each such pair may be a subset of atoms of the molecular system). In such a case, the energy of the molecular system may be rewritten as a sum of environment-dependent pair interactions.

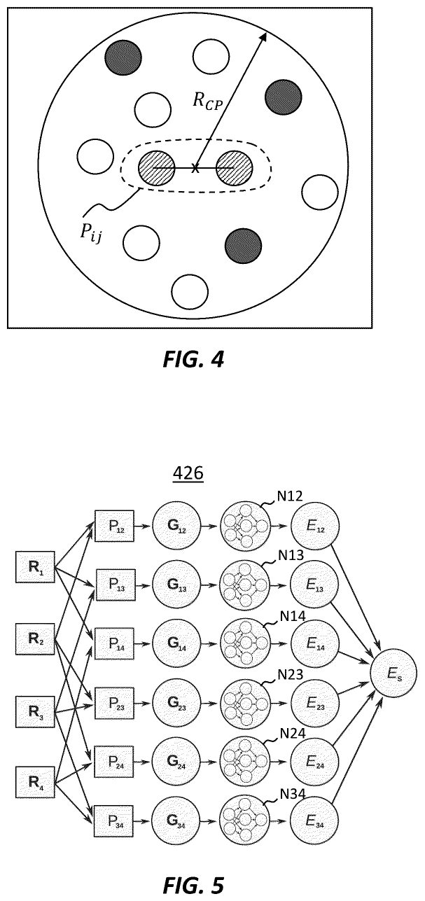

FIG. 4 shows an example of a neighborhood for a pair of atoms Pᵢⱼ. As shown in FIG. 4, atoms within the spherical neighborhood defined by a cutoff radius R_{CP} may be counted towards energy calculations when calculating energy associated with pair Pᵢⱼ. Note that R_{CP} may be determined based on a type of pair (e.g., R_{CP} radius may vary from one pair to another, and in general may not be selected to be the same as radius R_{C}). In some embodiments, R_{CP} may be selected to be, for example, about 6 Angstrom.

The expansion of atomic interactions into interactions of atom pairs offers a physically intuitive description of interaction energies and is related to the concept of the bond order. To explore the effect of such a pair approach on the accuracy of high-dimensional NN potentials, a CLM 426 implementation of CLM 126 may be used, as shown in FIG. 5 for a four-atom system. In the pair-based high-dimensional CLM 426, the structural information is decomposed into a set of atom pairs Pᵢⱼ (e.g., pairs P₁₂-P₃₄, as shown in FIG. 5) based on coordinates Rᵢ(e.g., R₁-R₄, as shown in FIG. 5), and their associated chemical environments Gᵢⱼ (e.g., G₁₂-G₃₄, as shown in FIG. 5), and the total energy of the system is constructed as a sum of the energy contributions Eᵢⱼ (e.g., E₁₂-E₃₄, as shown in FIG. 5) of these pairs obtained using neural networks Nᵢⱼ (e.g., N₁₂-N₃₄, as shown in FIG. 5). Similar to neural networks N₁ . . . N₄, as shown in FIG. 3, the neural networks N₁₂-N₃₄ may be different for different type of pairs (e.g., include different weights). It should be noted that selection of weights for the neural networks N₁₂-N₃₄ can be determined through training.

Even though individual neural networks such as neural networks N₁ . . . N₄ of CLM 326 or neural networks N₁₂ . . . N₃₄ of CLM 426 are configured to predict individual energies of atoms (as in the case of neural networks N₁ . . . N₄) or pairs of atoms (as in the case of neural networks N₁₂ . . . N₃₄), various implementations of CLM (e.g., CLM 326 or CLM 426) can be trained to obtain accurate corrections to the total energy results obtained using the MTM 124. In various embodiments, total energy may be calculated for a molecular system containing a few molecules (e.g., a dimer). The accuracy of the total energy (e.g., the total energy results obtained using MIM 124 with corrections from CLM 126) can be compared directly with a result obtained using quantum mechanical calculations. The training target energy correction can be implemented as an adjustment to the total energy computed by MTM 124. The correction may be computed as a difference between total quantum mechanical energy results and energy results using MIM 124 values.

In various embodiments, a method of generating a total energy for a molecular system includes the steps of (1) generating the atomic neighborhood descriptors (as described above) of the atomic neighborhoods or neighborhoods of atomic pairs, and (2) using these descriptors with an associated CLM (e.g., the CLM 326 for computing energies for individual atoms or the CLM 426 for computing energies for pairs of atoms) to obtain atomic (or atomic pair) energy values (e.g., the energy values E₁ . . . E₄ as shown in FIG. 3 or the energy values E₁₂ . . . E₃₄ as shown in FIG. 5).

In some implementations, when atomic pairs are used for determining total energy of a molecular system (e.g., an atomic pair may be a carbon-oxygen pair C—O), neural networks specific to such atomic pairs may be used. For example, a neural network N₁₂ may be specific to a particular type of pair C—O, (e.g., to an aromatic C—carbonyl O pair). Also, note that using pair specific neural networks can allow for smaller networks (e.g., networks with fewer layers) and can result in improved accuracy of the CLM 426. Further, using pair specific neural networks can result in generating improvements to the CLM 426 incrementally (e.g., CLM 426 may not need all the training data for all possible C—O pairs in chemistry to be trained for aromatic C—carbonyl O pair). Further, pairs such as aromatic C—carbonyl O pair are implemented within the MIM 124, and energy obtained using the MTM 124 for such a pair can be readily corrected using the CLM 426, when CLM 426 is configured to compute energy for the same type of pair. A further step of generating a total energy for a molecular system can include combining (e.g., summing) the individual energies from atoms or pair of atoms.

Descriptors are used for representation of a local atomic environment. Some neural networks realizations do not consider electronic interactions explicitly, but instead approximate the potential energy surface as a function of the atomic positions. Therefore, in building neural networks it can be important to obtain a proper representation of local atomic environments, or a model, which encodes relations between atoms and quantifies similarities and differences between atomic structures, thereby enabling mapping of atomic coordinates to a certain property, e.g., the total energy of the system. The quality of this mapping can be used to develop neural networks that are able to distinguish atomic structures and, therefore, to properly describe interatomic interactions.

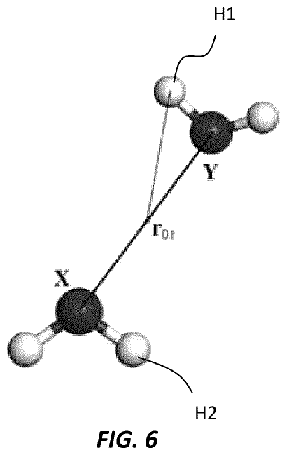

Similar to force field energies, neural networks can reflect the system symmetry with respect to global rotation/translations and permutations of atoms of the same element. Therefore, representations of the local atomic environment can be realized in terms of descriptors/fingerprints, or feature vectors with a fixed length, that preserve these symmetries. In various embodiments, descriptors are atom-centered, i.e., they are built for the local environment of a certain atom, (e.g., includes a set of few (10-50) atoms within a given cut radius R_{c} (as shown in FIGS. 2 and 4)). For non-covalent interaction, another approach may include pairwise representation concept, shown schematically in FIG. 6. For a given pair of atoms X and Y in different molecules/monomers a middle point r₀ᵢ of the X-Y distance can be set to be a reference point, and the nearest neighbors (e.g., atoms H1, and H2, as shown in FIG. 6) of atom X (or Y) in the first (or second) molecule can be considered (another possibility is to take into account atoms within the cutoff bonding distance r_{bond} from atoms X and Y in each molecule).

An example embodiment of a descriptor is simply the number of neighbors (the number of neighbors may be denoted as N) within some radius R of an atom A.

There are a few other properties that may be important and/or used for a suitable ‘input’ for neural networks for chemical purposes. The input may be invariant under the same changes that the energy is invariant under. For example, changing the order of counting the atoms in the neighborhood may not change the value of the input. Rotational invariance for inputs centered on atom A (rotating around atom A) may also be present. Conversely the input may not change when energy would change—e.g., shifting the positions of neighboring atoms may change the input. When the descriptors (input) are applied to a pair of atomic neighborhoods (A-B) they may be invariant with respect to the order of A and B. Thus, the total input to a computational model (e.g., to a CLM 126) is symmetrized.

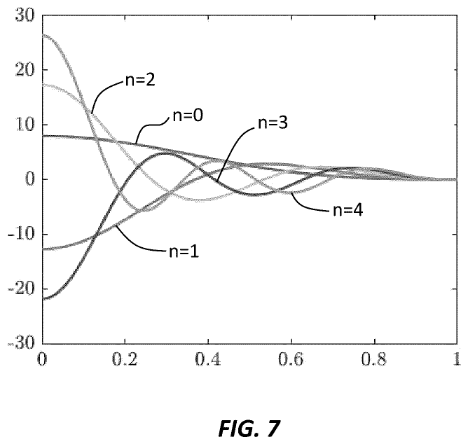

In some implementations, the symmetrization (herein also referred to as an encoding) may be done using atom-based symmetry functions (ABSF) (e.g., based on code stored in memory 120 and executed by processor 110 of FIG. 1). For example, when an energy value is associated with an atom (e.g., an atom may define a neighborhood with a cut off radius R_{C}) and the ABSFs are configured to be symmetric with respect to the permutation of the neighboring atoms of the same type. ABSF may be formed from (1) a set of radial functions describing the radial density of atoms in a neighborhood of A or a pair of atoms (e.g., a pair A-B), and (2) a set of spherical functions which describe the angular (or projected onto a sphere around A or pair A-B) distribution of surrounding atoms. They are also oscillating in angular space. FIG. 7 shows first four radial functions (n=1 . . . 4). The number nₘₐₓ of radial function used describes a degree of accuracy for approximating the neighbor density function. Similarly, the number of spherical functions lₘₐₓ used defines an angular distribution accuracy for approximating the neighbor density function.

The decomposition of the energy of a molecular system into energy contributions from pairs of atoms or ‘neighborhood-pairs’ of atoms is done by including, in the neighborhood of atom A of molecule A, atoms aᵢ of molecule A but also atoms bⱼ of molecule B. The same thing is then done for atom B of molecule B. This includes an additional symmetrization with respect to permutations between A and B. This approach is different from a typical technique of decomposition of the energy of a molecular system into energy contributions from single atoms, as that approach allows for adding energies for atoms A and B without including symmetrization with respect to permutations between A and B.

In various embodiments, a CLM (e.g., CLM 126) may be used to describe pair interactions between atoms A-B and estimate the intermolecular energy. The total energy of the molecular system may then be obtained by adding energy results obtained using CLM 126 and energy results obtained using MTM 124 (MTM 124 may include determining an energy of the molecular system associated with bonds such as energies associated with bond distortions, and/or the like).

In various embodiments, combining CLM 126 with MTM 124 may alleviate problems associated when CLM 126 is used without MIM 124, such as, for example, (1) predicting the energy of a molecular system having a collection of atoms and molecules in an external electric field, when CLM 126 was trained on input data (e.g., input data including location of atoms within the molecular system) that did not include effects of external electric field and (2) predicting the energy due to multi-body atomic interactions in the far range including effects of polarization, or dispersion. Far range interactions may require expanding the ‘neighborhood’ far enough so that such effects can be accounted. In the case of electrostatic interactions, the expansion may use very large neighborhoods, which may not be computationally achievable (or computationally efficient). Even to describe proper 3 and 4 body interactions (between molecules) the neighborhoods may become exceedingly large. Thus, MTM 124 provides a way to describe physical interaction between atoms in a molecular system using forces between atoms derived based on classical field description, while CLM 126 may be used for multi-body atomic interactions when classical description is difficult to determine explicitly.

In principle it is possible to correct a model describing the atomic interactions (e.g., the model may be MIM 124) using a CLM such as CLM 126. In some cases, CLM 126 may be used alone without MIM 124. However, using MIM 124 allows for incorporation of physical description when determining atomic interactions. Specifically, in some implementations, there are two physical features of a molecular system that a combined MIM 124 and CLM 126 model may describe. First is a proper modeling of molecular induction/polarization (e.g., the modeling of dipole moments in molecules in response to an external electric field). To model such a feature, a physics-based theory may be used (which may be formulated as a part of MM 124) and allows one to describe many-body (molecular) non-additive interactions based on relatively few benchmark calculations. If MIM 124 is not used for describing this physical feature and CLM 126 is used instead to describe this feature, the CLM 126 can be presented with (e.g., trained using) a very large number of trimers, tetramers, and the like to properly model the interaction, and the training input can be enlarged to accommodate such training examples. The second desirable physical feature is a relatively accurate description of electrostatic density and atomic interactions. In various embodiments, the atomic interactions may include (1) long-range atomic interactions, with at least some long-range atomic interactions not accounted for, or modelled, or truncated, to reduce computational complexity (e.g., the computational speed may be reduced from n² calculations to n·log(n) calculations). For example, when describing long range interactions, a Particle mesh Ewald (PME) method may be used. Note that in some cases, truncation of the long-range interactions using a simple (e.g., smooth truncation function) to zero at some distance may result in prediction errors, thus, a more complex truncation approach may be used. Additionally, or alternatively, a PME, or a fast-multipole method incorporating electrostatic interactions may be used instead of the truncation approach.

In some implementations, the process of determining a total energy of a molecular system may include the following steps and considerations:
1. augmenting/correcting atomic pair interactions (e.g., a pairwise interactions for which energy is determined using MIM 124) between groups of atoms (groups usually means molecules but not necessarily so).
2. The decomposition of the energy of a molecular system into energy contributions from pairs of atoms or ‘neighborhood-pairs’ of atoms can be done by including, in the neighborhood of atom A of molecule A atoms aᵢ of molecule A but also atoms bⱼ of molecule B. The same thing is then done for atom B of molecule B.
3. The atom-centered symmetry functions for molecular neighborhoods can be symmetrized with respect to interchange of A and B atoms to become pair-based symmetry functions.
4. The CLM 126 is split to give an independent energy for each molecule A and molecule B interaction for the pairs of atoms between molecule A and molecule B which is then summed as it can be in a molecular or Newtonian model for many-body expansion (first term). For example, the pair interaction between two H2O molecules (e.g., the first H2O molecule is the molecule with atoms labeled with letter A and the second H2O molecule is the molecule with atoms labeled with letter B) would be H1A-H1B+H1A-OB+H1A-H2B+OA-H1B+OA-OB+OA-H2B+H2A-H1B+H2A-OB+H2A-H2B.
5. The output of a neural network (e.g., CLM 126) is fitted to the residual error of the starting physical model (e.g., the starting physical model may be MTM 124). The residual error can be a difference between MTM 124, and the results based on quantum mechanical calculations (e.g., quantum mechanical calculations for a dimer energy). In case MIM 124 is not used, the CLM 126 is fit to the results based on the quantum mechanical calculations. In various embodiments, there could be tens of thousands or more of dimers generated and computed for training CLM 126. In various embodiments, CLM 126 may be used to correct results of MTM 124 for 2-body interactions, while contributions to the total energy of the molecular system from other multi-body interactions may be predicted using MIM 124.
6. The corrections using CLM 126 can be truncated at some intermediate distance away from an atom (or a pair of atoms), since after some distance the physical model MIM 124 may be accurate after that distance.
   a. In various implementations, radial symmetry functions approach zero at a sufficiently large distance. In an example implementation of CLM 126, a layer may be inserted into the CLM 126 that is configured to suppress the energy of that interaction to zero at some distance with a smooth cross-over.
   b. In addition to making the CLM 126 well-behaved, this also helps the separability of the model—i.e., atoms far away do not try to ‘help’ improve the total energy, and therefore the fitted pair interaction (correction in this case) can be lifted out and used in a similar situation with different far away atomic constituents.
7. The energy corrections from CLM 126 may be pair-specific, i.e., not decomposable. Thus, the pair of two atoms can be treated as a single unit (similar to how an atom is treated). For example, electrostatic energy may be determined as a combination of individual contributions of energy terms from two atoms forming a pair, while other atomic interaction of two atoms forming a pair may not be represented using separable energy terms.
8. The pair interaction may be indexed by a chemical type, as described above. For example, a pair of C—O may have a chemical type of aromatic C—carbonyl O pair. The indexing by type allows for correct specific interactions for each type and allows to not have to obtain a QM training set for all of chemical space. Also, indexing by type allows to have a relatively light-weight CLM 126 for describing atomic interactions ((Input size:20:20:1) with a ReLu or Tan h activation function (or other desired activation function)).
9. In an example embodiment, (and this is partially responsible for the improved precision of the approach of combining CLM 126 and MIM 124) the non-bonded interactions (i.e., interactions between molecules) are corrected separately using CLM 126 while bonded interactions may be described by MTM 124 (note that bonded interactions may be corrected in the same manner). Such approach may overcome various difficulties associated with modeling atomic bonds using CLM 126. Using CLM 126 for describing (inter- or intra-molecular interactions) the much stronger intermolecular and atomization predictions are then described by MIM 124, as CLM 126 may use large computational resources to describe such interactions. It should be noted that in some cases, the bonded interactions may also be described using a suitable computer learning model, however, a computer learning model for bonded interactions may be trained differently than a computer learning model for non-bonded interactions (e.g., due to energies associated with bonded interactions being much larger than energies associated with non-bonded interactions). By training CLM 126 to determine non-bonded interactions for different atomic types while describing bonded interactions using MIM 124, the computational time may be significantly reduced.
10. Further, in some implementations, for example:
   a. the range of inputs for relative orientation of dimers/pair neighborhoods and for the deformation of each monomer (and this influences intermolecular energy) can be different. Relative orientations cover the whole Euclidean group, whereas bonds and angles vary only very slightly about their equilibrium bonded values. Thus, the bonded distortions can be rescaled to amplify their fluctuations and add a separate set of descriptors where these amplified values are hashed. This allows to keep a lower overall set of descriptors (e.g., lower orders of the oscillations in FIG. 7).
   b. the forces can be obtained from the CLM 126 by extracting such forces via package derivative functionality associated with CLM 126, which is used for training the CLM 126. Further, the derivatives of the descriptors may be determined analytically. Also, the chain rule is used to obtain the total forces acting on various atoms of a molecular system. The movement of atoms may then be described via a molecular dynamics techniques such as Verlet or Leap-frog techniques, and/or the like.
   c. the MIM 124 and the CLM 126 may use either fixed precision arithmetic or other suitable float point arithmetic that can be performed by one or more CPUs executing the MIM 124 and the CLM 126.

The atomic neighborhood (in the many senses of the word, but roughly proportional to ‘bond-distance’) is responsible for the splitting of the interaction determining designation (first splitting is chemical element i.e., O, H, N etc.) into subtypes (i.e., aromatic carbon, aliphatic carbon, etc.). In some embodiments, the sub-type splitting and the correcting neural network are typed in the same manner as the underlying Newtonian model (except, for example, pair-specific and not decomposable). In some implementations, a pair-specific term is roughly representing charge transfer. In some implementations, instead of having a C—O pair interaction and correction a (Aromatic C)-(carbonyl O) pair interaction and correction is defined. It is conceivable that the ‘typification’ can be done in substantially real-time: i.e., there can be inputs that represent the neighborhoods of atoms a and b and therefore adjust the network behavior to shift between types as an integral object. This may increase the complexity of the network and reduce the sensitivity to intermolecular interactions.

Additionally, as typed pair interactions have been made in the underlying model, the same typed corrections can be applied. The typification decision can be human, or algorithmic (e.g., automatically done by processor 110); but in some embodiments it is done a-priori. Note that this implies that molecules cannot disintegrate/break bonds—as it will change the atomic neighborhoods. As above, the decision to stay in the inter-molecular energy range and interactions allows a direct fit to that energy range and therefore permits (much) greater accuracy.

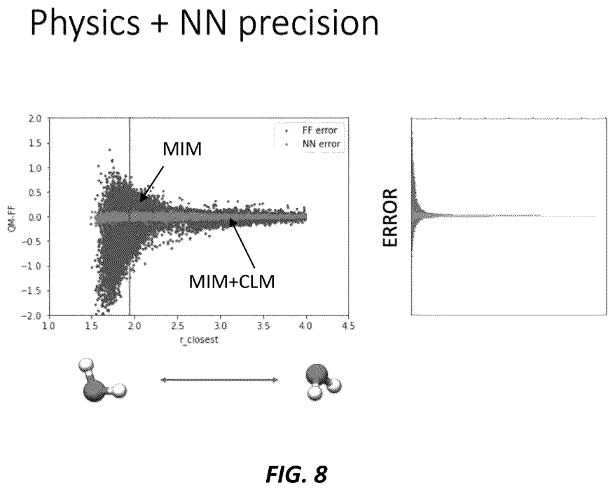

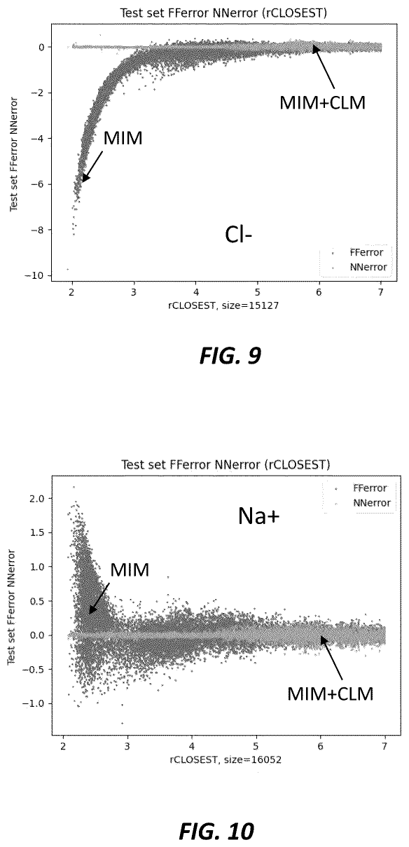

FIGS. 8-10 show data points indicating an error in a force computed between various molecules (e.g., FIG. 8 shows energy errors for water dimer) and FIGS. 9 and 10 show energy errors for H2O—NaCl. FIG. 9 shows force between a water molecule and a negative Cl ion, and FIG. 10 shows a force between a water molecule and a positive Na ion. Note, as shown in FIGS. 8-10, when combining MIM 124 and CLM 126, the resulting error is substantially reduced. For instance, at short distances the correction due to CLM 126 significantly improves the accuracy of the force calculations. In FIGS. 8-10, the X axis is distance between molecules, and the Y axis is the ERROR from force calculations obtained using quantum mechanical calculations.

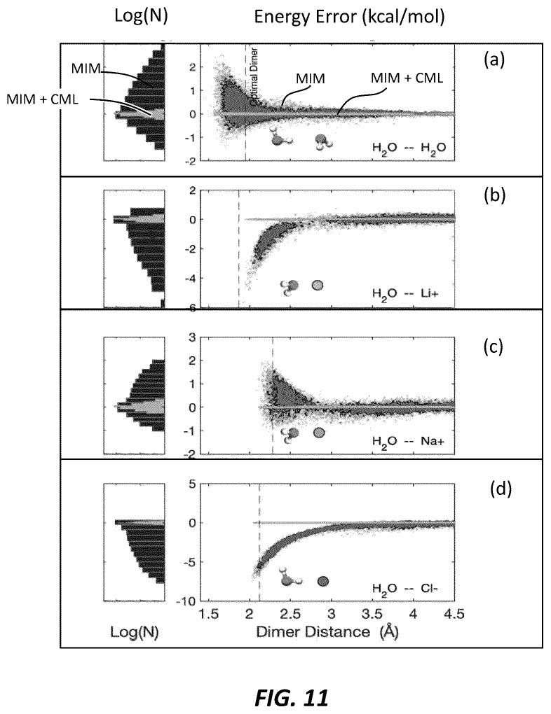

FIG. 11 shows energy error for molecular interaction when using MIM 124 with and without CLM 126. FIG. 11 (a) shows a molecular interaction for water-water molecules, FIG. 11 (b) shows a molecular interaction for water-lithium+ cation molecules, FIG. 11 (c) shows a molecular interaction for water-sodium+ cation molecules, and FIG. 11 (d) shows a molecular interaction for water-chlorine⁻ anion. The histograms are in log units to amplify the distributions. The histograms indicate the number of molecular configurations (e.g., various orientations of water dimer) leading to a depicted energy error. In the strongly interacting region of close approach, the analytical model (e.g., MIM 124) has significant errors>5 kcal/mol. However, combination of MIM 124 and CLM 126 has a good accuracy for various distances. For example, the accuracy may be such that the energy error may be less than a few kcal/mol, less than a fraction of kcal/mol, or in some cases, less than a few hundredth of kcal/mol.

As shown in FIG. 11, the augmentation of 2-body interaction energies with computer learning models (e.g., models based on neural networks) may result in a significant improvement. The plots corresponding to errors when molecular interactions are calculated using the combined MIM 124 and CLM 126 show almost no error (herein, the error is calculated as a deviation of hybrid model energies from the corresponding QM ones). When combining MIM 124 and CLM 126, interactions described by CLM 126 are related to the residual error of the MIM 124. In each case the accuracy of the hybrid model is substantially equal to that of QM methods at the highest level of theory.

In various embodiments, combination of MIM 124 and CLM 126 allows one to preserve at least some analytical physical description of intermolecular interactions. For instance, the proposed combination of an analytical polarizable force field model (MIM 124) with an intermolecular 2-body neural network short-range correction (CLM 126) achieves accurate results while also describing effects of polarization using analytical expressions used in MIM 124. These analytical expressions, for example, for the polarization, allows one to parameterize the model on QM-computable sets of molecules (e.g., dimers), and ensures that the model is correct in describing large numbers of molecules. The analytical terms of MIM 124 allow the intermolecular hybrid model to naturally and properly respond to external perturbations such as an applied electric field. These terms also allow for the full intermolecular hybrid model to be properly truncated at long-range, which is used for efficient and accurate simulation. In the regime of close molecular approach, however, where many simplifying assumptions are used to represent Quantum Mechanics with Newtonian modes-predictions obtained using MIM 124 may become inaccurate, thereby correction using CLM 126 may be desirable. CLM 126 is designed to be computationally efficient (e.g., by training smaller NNs, which characterize the interaction of molecular pairs into pairs of chemical types), readily trainable, and well-behaved even when trained with limited data.

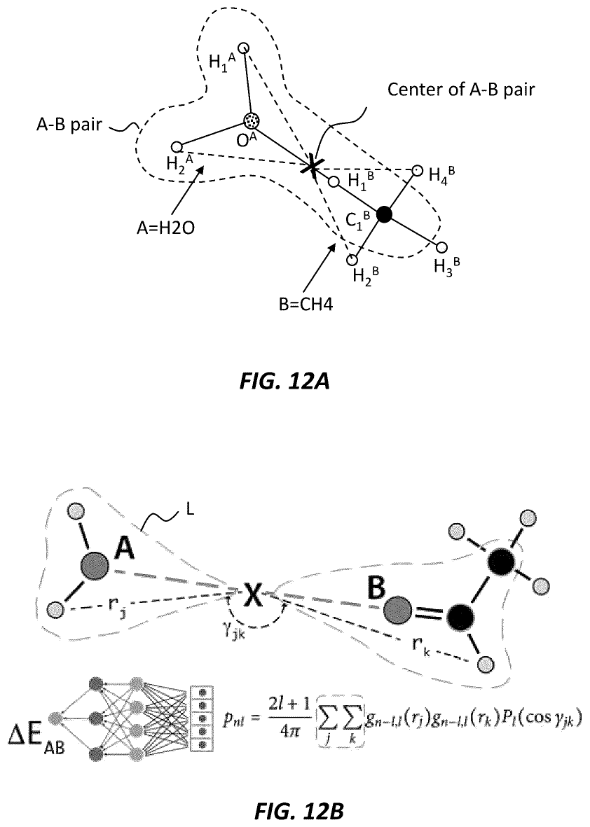

FIG. 12A shows interaction between molecule A of water and molecule B of methane (herein, such system is referred to a molecular system A-B). Molecule A of water includes atoms labeled as O^{A}, H₁^{A}, H₂^{A}, and molecule B of methane includes atoms labeled as C^{B}, H₁^{B}, H₂^{B}, H₃^{B}, H₄^{B}. As shown in FIG. 12A, for pair of atoms O^{A}-H₁^{B} neighboring atoms include H₁^{A}, H₂^{A}, and C^{B}, H₂^{B}, H₃^{B}, H₄^{B}, and these atoms are used for determining symmetry functions function G for pair of atoms O^{A}-H^{B}. The symmetry functions G is computed about a symmetry point X (as shown in FIG. 12A located as a mid-point between atom O^{A} and H₁^{B}. When computing G, locations, of the neighboring atoms may be considered. Further, while FIG. 12A shows a neighborhood for one particular pair O^{A}-H₁^{B}, other possible atomic pairs between molecule A and molecule B can be considered for determining the total energy of the molecular system having molecule A and molecule B. For example, pairs such as {Hᵢ^{A}, Hⱼ^{B}},{O^{A}, Hⱼ^{B}},{Hᵢ^{A}, C^{B}}, and {O^{A}, C^{B}}, may be considered. In some cases, interactions for pairs between atoms with a large separation (e.g., for a pair {H₁^{A}, H₃^{B}} may not be considered, as such interactions have a relatively low contribution to the overall energy of the molecular system A-B).

FIG. 12B shows another representative diagram of an interacting pair of a molecule A (e.g., water) and a molecule B (e.g., acetaldehyde). A neural network (e.g., CLM 126) may be used to augment the intermolecular interactions described by MIM 124. As described above, in contrast to the prevailing practice of partitioning the potential energy onto individual atoms, the energy may be partitioned for energy corresponding to interacting pairs of atoms (A-B). Such partitioning may be achieved by using point-centered symmetry functions centered at the midpoint X between A and B (A-X-B). The atoms included into the fingerprint summations are neighbors of both A and B. Either a bond-distance (e.g., d=1 for including nearest bonded neighbors of A and B, or d=2 for breaking the rotational symmetry of the descriptors of terminal atoms) or a cutoff distance (neighbors of A and B within some cutoff distance) may be used as a criteria for neighborhood membership. The midpoint construction geometrically symmetrizes the interaction fingerprint with respect to the permutation A<->B. This fingerprinting couples the intermolecular energy to the relative orientation as well as to the monomer distortions of the two molecules. The symmetry function summations are over neighborhoods of both A and B, which is shown in FIG. 12B by dotted line L. The fingerprint is provided and/or input to an (AB) specific neural network to produce an energy correction to the MIM interaction energy between A and B (ΔE_{AB}). The interaction fingerprint is produced by expanding the atomic density neighboring X via an orthonormal basis set, including spherical Bessel functions for the radial basis, and the usual spherical harmonics (Legendre polynomials) for the angular triples atom-X-atom. The midpoint construction is computationally economical as the terms in the summation contain X.

As analytical force-field errors arise mostly at close range, the corrections described by a neural network (NN) model (e.g., by CLM 126) may be tapered beyond a chosen distance between atoms A and B (e.g., such distance may be for example a few angstroms (Å) such as 4, 5, or 6 Å, or the like), beyond which the interactions are computed by MTM 124. Rather than relying on the built-in decay of radial symmetry functions, a Tensorflow lambda layer may be appended to CLM 126 to smoothly force the outputs to zero beyond the cut-off distance.

Like most analytical Force-Fields, the NN correction for the pair of atoms is further subdivided by their atomic types. For example, instead of encoding a general interaction correction NN model for, for example, Carbon-Nitrogen, the interaction corrections can be more specific in a NN model for, for example, aromatic Carbon-Amide Nitrogen. This typification can provide advantages, such as, for example, it breaks up the full chemical interaction space into much smaller training groups so that model improvement is realized in segments and avoids a pre-compute of a large number of possible intermolecular interactions. As a consequence, the resulting total hybrid accuracy of representation of the interaction can be substantially higher. In some cases, the accuracy may be on the order of convergence accuracy of the QM method chosen for the training set. Moreover, the limited amount of encoded information permits a small and therefore fast neural network. Because the fingerprint itself does not encode the chemical environment but the relative orientation and bonded distortion of the interaction, it is also much smaller and more computationally efficient. Moreover, it can be convenient to have the NN correction have the same typification as the underlying analytical force field model.

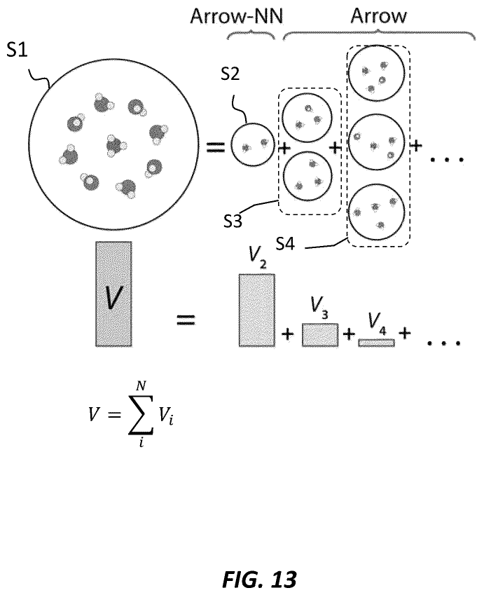

FIG. 13 graphically shows how MIM 124 and CLM 126 may be combined to model molecular interactions. For example, FIG. 13 shows a system S1 of nine molecules. Interactions within system S1 using MIM 124 and CLM 126 may be decomposed by determining interactions for subsystems having at least some of the molecules of system S1. For example, interactions may be determined for a subsystem S2 having two neighboring molecules using, for example, CLM 126 model, and then determining interactions for subsystems S3 having three molecules, and for subsystems S4 having four molecules, and the like. Various interactions for various subsystems then may be added resulting in overall description for molecular interactions for system S1.

### Examples of Descriptors

In recent years various descriptors have been proposed based on local PES, the local atomic density, the radial/angular distribution functions, etc. In some implementations, a descriptor can:
(a) exhibit the symmetries of the potential energy,
(b) use minimal manual fine-tuning,
(c) provide a parameter for adjusting its resolution,
(d) be continuous and differentiable, so that analytical forces can be obtained,
(e) be computationally efficient, and
(f) not scale with the number of chemical species.

The choice of descriptors can depend strongly on the atomic system under consideration, as well as available computational resources.

In one embodiment, the spherical Bessel descriptors based on the concept of the atomic density are applied [SB]. The local atomic environment around a given point r can be described with atomic neighbor density function:

$${ρ}^{k} ( r ) \approx {\sum }_{j} {w}_{j}^{k} δ ( r - {r}_{ij} ) , \qquad ( eq . 1 )$$

where rᵢⱼ is the relative position vectors of each neighbor j with respect to the reference point r₀ᵢ, wₖⱼ is the weight factor that can be used to distinguish the k^{th} species in a multicomponent system (in expressions below it is set to be 1). Expanding this density over orthonormal basis functions of full sphere with radius r_{cut} yields:

$$ρ ( r ) \approx {\sum }_{n = 0}^{{n}_{max}} {\sum }_{l}^{n} {\sum }_{m = - l}^{l} {C}_{nlm} {g}_{n - l , l} ( r ) {Y}_{lm} ( θ , φ ) , \qquad ( eq . 2 )$$

where gₙₗ(r) are radial basis functions and Yₗₘ(Θ,φ) are spherical harmonics, while nₘₐₓ specifies the order of the approximation. The expansion coefficients cₙₗₘ for the i^{th} atomic pair X-Y (therefore, with respect to the point r₀ᵢ={rᵢ,qᵢ,jᵢ}), are obtained from the relative spherical coordinates {rᵢⱼ,qᵢⱼ,jᵢⱼ} of the nearest neighbors of atom X of the first molecule and atom Y in the second molecule as shown in (FIG. 6):

$${c}_{nlm} = {\sum }_{j} {g}_{n - l , l} ( {r}_{ij} ) {Y}_{lm}^{*} ( {θ}_{ij} , {φ}_{ij} ) . \qquad ( eq . 3 )$$

These coefficients are not invariant with respect to rotation, but the power spectrum calculated as:

$${p}_{nl} = {\sum }_{m = - l}^{l} {c}_{nlm}^{*} {c}_{nlm} = \frac{2 l + 1}{4 π} {\sum }_{jk} {g}_{n - l , l} ( {r}_{ij} ) {g}_{n - l , l} ( {r}_{ik} ) {P}_{l} ( cos {γ}_{jik} ) , \qquad ( eq . 4 )$$

where Pₗ(x) is the Legendre polynomial of order l, includes real-values numbers, is invariant to translation, rotations and inversion as well as to permutations of the same atoms and can be used as descriptors.

In some implementations, various functions can be chosen for gₙₗ(r). It is shown [SB] to be efficient to use a linear combinations of two spherical Bessel functions jₗ(r) of the first kind as follows:

fₙₗ(r)=aₙₗjₗ(ruₙₗ/r_{cut})+bₙₗjₗ(ru_{n+1,l}/r_{cut}), (eq. 5)

where uₗₙ is the (n+1)^{th} non-zero root of jₗ(r), and r_{cut} is the cutoff radius. With a special relation for coefficients aₙₗ and bₙₗ these functions can be made twice differentiable even at the cutoff radius. The Gram-Schmidt orthogonalization procedure, applied sequentially to fₙₗ(r) for 0<n£nₘₐₓ, results in a set of orthogonal and normalized radial functions gₙₗ(r). The main advantage of the described approach is the continuity and twice differentiability of gₙₗ(r) within the sphere of the cutoff radius. In addition, this infinite orthonormal basis set gₙₗ(r) is complete for square-integrable functions with the given boundary conditions, and in general its completeness makes training with neural network become more steady, reliable, and predictable.

The approximation order in the described approach can be determined with the parameter nₘₐₓ, and the number of generated descriptors N_{d} is (nₘₐₓ+1) (nₘₐₓ+2)/2. The low number of descriptors do not describe properly the atomic environment, and feature vectors might contain insufficient information about the system. The increase in N_{d} enhances the descriptor resolution and, therefore, the accuracy of the atomic environment representation, but molecular dynamics simulations with the large number of descriptors use substantial computational resources.

The cutoff radius r_{cut} is an important parameter of the local atomic environments and generated descriptors. Small r_{cut} are insufficient to properly describe the atomic environment. For atom-centered representations, increasing the value of r_{cut} implies the inclusion of additional atoms into the local environment and, therefore, uses more computational resources. As a result, in some implementations, it can be considered efficient to set the cutoff radius as small as possible.

Consistent with disclosed embodiments, FIG. 14 shows a method 1401 of determining an energy for a collection of atoms. The method 1401 includes step 1411 of determining a first plurality of atomic interactions between a first set of atoms and a second set of atoms, based on molecular interaction model (MTM) such as MTM 124, as described above in relation to FIG. 1. The first set of atoms may be a set of atoms forming a first molecule, and a second set of atoms may be a set of atoms forming a second molecule.

Further, the method 1401 includes step 1413 of providing a plurality of inputs related to the first set of atoms and the second set of atoms to a neural network model (NNM) (e.g., NNM may be CLM 126 as described above, herein and elsewhere it may be also referred to as dENN) formed from a plurality of neural networks (e.g., neural networks N1 . . . N4, or N₁₂ . . . N₃₄, as shown in FIGS. 3 and 5) to determine the second plurality of atomic interactions.

Additionally, following the step 1413, the method 1401 includes step 1415 of training the NNM to reduce error between (1) the combination of the first plurality of atomic interactions and the second plurality of atomic interactions and (2) atomic interactions obtained using quantum mechanical calculations.

Further, the method 1401 includes step 1417 of modifying the MIM to reduce error between (1) the combination of the first plurality of atomic interactions and the second plurality of atomic interactions and (2) atomic interactions obtained using quantum mechanical calculations.

Further, the method 1401 includes step 1419 of receiving a third set of atoms, step 1421 of inputting the representation of the third set of atoms to output a prediction associated with an energy or a state of the third set of atoms, and step 1423 of sending a signal to a user to present the prediction. In various embodiments, the predictions are obtained using a combined MIM and NNM (e.g., the combined MIM 124 and CLM 126).

In various embodiments, the method 1401 includes predicting at least one of the partition function, free energy, enthalpy, entropy, density, hydration free energy, conductivity, heat of vaporization, diffusion properties, binding free energy, proportion of reactants, or non-equilibrium, or combination thereof of properties of the third set of atoms. Additionally, or alternatively, the method 1401 can be used to predict any other suitable physical quantity that can be derived from the computations of potential energy between the third set of atoms.

As described herein, the method 1401 is configured to combine MIM and NNM models in any suitable way to achieve accurate predictions. In one implementation, of the method 1401, the combination of MIM and NNM is realized by adding the output of the first plurality of atomic interactions (i.e., the interactions obtained using MIM) and the output of the second plurality of atomic interactions (i.e., the interactions obtained using NNM).

Further, the method 1401 may include smoothly, over a predefined interval, switching the output of the NNM to zero at a predefined distance between the first set of atoms and the second set of atoms. In some cases, the smooth switching to zero of the output of neural networks is realized by a neural network analytical layer such as a lambda layer in TensorFlow.

In an implementation of the method 1401, the combination of MIM and NNM is realized by using a substitution of at least one parameter of the MIM by an output of one of neural network models used to form NNM (e.g., neural network model N₁, as shown in FIG. 3).

Moreover, in an implementation of method 1401, the combination of MIM and NNM is realized by applying the first plurality of atomic interactions and the second plurality of atomic interactions to a first subset of atoms in the third set of atoms and a faster computational method (e.g., MIM) to a second subset of atoms in the third set of atoms. In an example embodiment, the second subset of atoms may not overlap with the first subset of atoms. Further, in some cases the first subset of atoms and the second subset of atoms may be complimentary. In some cases, when using method 1401, the first set of atoms includes only a first single atom and the second set of atoms includes only a second single atom.

In some implementations, the first set of atoms includes a first atom and at least one neighbor atom of the first atom and the second set of atoms includes a second atom and at least one neighbor atom of the second atom.

In some cases, the first set of atoms and the second set of atoms are within at least a portion of a common molecule.

Further, in some embodiments, the first set of atoms is the same as the second set of atoms.

In some cases, the first set of atoms include atoms within a first molecule and the second set of atoms include atoms within a second molecule different from the first molecule.

In various embodiments, as described above, a neighborhood is defined by a predetermined distance from a central atom or atoms or from a geometrically assigned interaction center, or a predetermined number of bonds away from a central atom or atoms. In some implementations, for example, the geometrically assigned interaction center can be a reference point and is located at the center of a line connecting the first atom and the second atom. A geometrically assigned interaction center for a group of atoms may be also a centroid for such a group, when there are more than two atoms in that group. Further a center atom may be an atom that is closest to such a centroid. If more than one atom is located the same distance away from the centroid, than any one of such atoms may be selected as a central atom. Additionally, a neighborhood may be defined for any selected atom of a molecular system. For example, for a water molecule interacting with NaCl, an O atom may be selected to define a neighborhood about that atom. Additionally, or alternatively neighborhoods about Na and CL may be defined. Further, in some cases, a neighborhood about a pair of atoms may be defined (e.g., a neighborhood about a pair of O-CL or O—Na may be defined). In some cases, a neighborhood about a set of atoms may be defined. For example, a neighborhood about a center of a unit cell containing a few atoms within a crystal structure may be defined.

In various embodiments, the representation of the at least one of the first set of atoms or the second set of atoms includes at least one of an orientation of an atom from the first set of atoms or the second set of atoms, a neighborhood of an atom from the first set of atoms or the second set of atoms, a type of an atom first set of atoms or the second set of atoms, or a relative location of an atom from first set of atoms or the second set of atoms.

The representation of at least one of the first set of atoms or the second set of atoms may have a predetermined length not dependent on a number of atoms in the at least one of the first set of atoms or the second set of atoms.

The representation of the at least one of the first set of atoms or the second set of atoms may have a predetermined length not dependent on a rotation, a translation, or a reordering of member atoms in the at least one of the first set of atoms or the second set of atoms, or reordering of the sets themselves.

In various embodiments, the NNM may include the plurality of neural network models each one of the neural network models configured to describe either a distinct atom type or a distinct atomic pair type. An example atomic pair type is a pair of atoms with each atom being of a particular type. For example, an atomic pair type may be a pair formed by an aromatic carbon and an amine nitrogen instead of a carbon nitrogen pair, or a pair formed by an aromatic carbon and an ester oxygen instead of a carbon oxygen pair, and/or the like.

Further, in various embodiments, the plurality of atom types are inferred from the membership of an atom or pairs of atoms in the periodic table, and the chemically designated atoms in the neighborhoods of an atom or a pair of atoms. Furthermore, in some cases, an atomic type of an atom may be inferred based on its location in the periodic table (e.g., its associated chemical element), topological position of its neighboring atoms, neighboring atoms' position in the periodic table (e.g., the neighboring atoms associated chemical elements), and in some cases, neighboring atoms' associated atomic types. Similarly, atomic type for a pair (or a group) of atoms may be inferred based on the atoms' associated chemical elements in the pair (or the group) of atoms, topological position of neighboring atoms of the pair (or the group) of atoms, as well as associated chemical elements of neighboring atoms of the pair (or the group) of atoms, and in some cases, associated atomic types of neighboring atoms of the pair (or the group) of atoms.

In an example embodiment, the plurality of atom types includes an aromatic carbon, an aliphatic carbon, a carbonyl oxygen, and an ether oxygen, or, for example, a hydroxyl oxygen.

In various cases, the interactions between the first set of atoms and the second set of atoms may be computed using quantum mechanical methods and include one or more collections of interactions of atoms within a single molecule, atoms of pairs of molecules, atoms of triples of molecules, atoms of a molecule and an ion, atoms of a molecule and a charge, and/or atoms of a molecule within an applied electric field. The interactions between the first set of atoms and the second set of atoms computed using the quantum mechanical methods include at least one sub-component of the description of the collection of atoms computed by quantum mechanics. The sub-component may be an energy sub-component such as, for example, Dispersion, Exchange-Repulsion, Electrostatic, or Induction Interactions. The sub-component may also be the effective energy change produced by including Nuclear Quantum effects, and polarization induced by external fields and molecules.

In various embodiments, an MIM (e.g., MIM 124) may be used to describe electrostatic response interactions, which may include at least one of induction, polarization or charge transfer. MIM may take as an input at least one of a monopole, dipole, quadrupole, or octupole having a radial accuracy within about one percent, about five percent, about ten percent, about fifteen percent, about a few percent or about a few tens of percent points.

The MIM 124 may be modified (e.g., various parameters of the MIM 124 may be suitably adjusted) to reproduce experimentally observable or measurable macroscopic properties of collections of molecules. For example, the parameters of the MIM 124 may be adjusted to reduce the error between the outputs of MIM 124 and atomic and molecular set properties computed by quantum mechanical methods. The outputs may include energies of interaction between molecules, partial charges, induced charge distribution changes, or correlated charge fluctuation responses. In some cases, the adjustment of the parameters includes fitting at least one of the induction response, the non-additive energies, or various components of a total energy to obtain an accurate prediction for the molecular and/or atomic polarization. Herein, the term “induction response” can refer to the polarization of atoms in a molecule in response to an external electric field. Induction response can arise due to the movement of electrons in a molecule when an external electric field is applied. This movement causes a redistribution of electron density in the molecule, which can lead to the formation of induced dipoles. The magnitude and direction of the induced dipole depend on the strength and orientation of the external electric field and the polarizability of the atoms in the molecule. The induction response may be accounted for in molecular interaction model calculations by introducing an additional term that accounts for the interaction between the induced dipoles in neighboring atoms. This term is known as the “polarization energy” and is added to the non-bonded interaction energy between the atoms. The polarization energy is proportional to the square of the induced dipole moment and the strength of the external electric field. Thus, the induction response is the polarization of atoms in a molecule in response to an external electric field. This effect is important in molecular interaction model calculations as it can affect the stability, conformation, and properties of molecules. To account for induction response, the molecular interaction calculations can include a polarization energy term that accounts for the interaction between induced dipoles in neighboring atoms. The energy prediction may be obtained by computing an energy of a molecule probed by a charge (or an applied electric field) at various positions around the molecule.

The MIM 124 may be truncated at long ranges. For example, truncating the molecular interaction model at long ranges includes using a method such as a Particle Mesh Ewald or Fast

### Multipole Method.

Consistent with disclosed embodiments, FIG. 15 shows a method 1501 of determining an energy for a collection of atoms. The method 1501 includes step 1511 of determining, at a processor, a first plurality of atomic interactions between a first set of atoms and a second set of atoms using an analytical molecular interaction model (MIM), which may be the same or similar to MIM 124.

Further, the method 1501 includes step 1513 of providing a plurality of inputs to a plurality of neural network models (NNM) (e.g., NNM may be CLM 126 as described above) formed from a plurality of neural networks (e.g., neural networks N1 . . . N4, or N₁₂ . . . N₃₄, as shown in FIGS. 3 and 5) to determine a second plurality of atomic interactions between the first set of atoms and the second set of atoms, each input from the plurality of inputs being a representation of at least one atom of the first set of atoms or the second set of atoms.

Additionally, following the step 1513, the method 1501 includes step 1515 of training the NNM by reducing an error between (1) an interaction produced by a combination of the first plurality of atomic interactions and the second plurality of atomic interactions and (2) an interaction between the first set of atoms and the second set of atoms computed using quantum mechanical methods.

Further, the method 1501 includes step 1519 of receiving a representation of a third set of atoms, step 1521 of inputting the representation of the third set of atoms into MIM and NNM to output a prediction associated with an energy or a state of the third set of atoms, and step 1523 of sending a signal to a user to present the prediction. In various embodiments, the predictions are obtained using a combined MIM and NNM (e.g., the combined MIM 124 and CLM 126).

Example Molecular descriptors

In various embodiments, when predicting intermolecular interactions, binding and solvation free energies can be accurately determined. Intermolecular interactions may be sensitive to geometries of intermolecular contacts. Therefore, to fit QM dimer interaction energies in CLM 126, descriptors may be assigned to each intermolecular atom-atom contact within a certain distance cutoff (e.g., a distance cutoff may be a few Angstroms, such as 4, 5, or 6 Angstroms). These descriptors differ from commonly used atom centered descriptors describing the environment of each atom in the molecular system and, thus, are more appropriate for accurate fitting of intramolecular energies and describe properties such as torsional barriers and vibrational spectra. Descriptors describing an atom-atom contact (an intermolecular bond) can include 2/rᵢⱼ function (rᵢⱼ—the distance of the contact) and sum of terms dependent on relative coordinates of atoms of the contact and environment atoms covalently bound to atoms of the contact versus geometrical center X̅ of the contact. The sum of terms may be given by expression similar to (eq. 4) described above:

$${p}_{nl} = \frac{2 l + 1}{4 π} {\sum }_{jk} {g}_{n - l , l} ( \vert {\overline{r}}_{j} - \overline{X} \vert ) {g}_{n - l , l} ( \vert {\overline{r}}_{k} - \overline{X} \vert ) {P}_{l} ( cos {γ}_{jk} ) \qquad ( eq . 6 )$$

where 0≤n≤nₘₐₓ, 0≤l≤lₘₐₓ,
Pₗ is the Legendre polynomial of order l and γⱼₖ is the triplet angle between atoms i, j and k, radial basis functions gₙₗ(r) are linear combinations of spherical Bessel functions of the type

$${j}_{l} ( r \frac{{u}_{ln}}{{r}_{c}} )$$

with expansion coefficients chosen to satisfy the conditions of orthogonality and zero first and second derivatives of gₙₗ(r) vs r at the cutoff radius r_{c}. By construction the descriptors can be invariant to permutations of atoms of the atomic contact and environment atoms. In some implementations, spherical Bessel descriptors with nₘₐₓ=lₘₐₓ=6 may be used.

### Example Training Dataset Generation

The CLM 126 corrections to the MIM 124 may be trained on multiple conformations of molecular dimers. To cover the space of intermolecular orientations, several procedures may be combined to generate dimer geometries. For example, grid, liquid MD dimers (e.g., dimers in a liquid molecular system) and vacuum MD dimers (e.g., dimers in a gaseous molecular system) can be combined. For a given molecular dimer, a regular grid of molecular conformations can be generated, for example, by translating one monomer vs another along the line connecting their centers of mass with a step of 0.2 Angstrom, then rotating each monomer in spherical angles θ and (p with a step of 10 degrees.

In some cases, internal geometries of monomers may be kept fixed (e.g., for minimal energy geometries). In some cases, liquid MD dimers for molecules A and B are sampled from MD simulations of A solvated in a box of B monomers and vice versa. Extracted dimer geometries have the closest monomer distance in the range of 1.5 to 5 A. MD dimers are also filtered to be close to the grid dimers within a certain threshold of root-mean-square deviation (RMSD) value. The latter is chosen in such a way that the coverage of grid dimers by MD dimers is no less than 80%. To ensure a uniform sampling of grid dimer space by MD dimers, a cap is set that limits a number of MD dimers that are associated with each grid dimer.

In various cases, the purpose of vacuum MD dimers is to augment the liquid MD dimers by supporting sampling of the conformational space that is either not sampled by grid dimers and/or by liquid MD dimers before RMSD filtering. Vacuum MD dimers can be generated by running MD simulations of molecule A in a box of few molecules B, but the non-bonded interactions between A and B are, for example, either completely removed or reduced down to 1-10%. The latter is done, for example, by running TI annihilation simulation for lambdas=0.9-0.99. Then dimers are extracted from these simulations. To use relevant dimers, the vacuum dimers are filtered by computing dimer energies using MTM 124 and leaving the dimers for which dimer energy is less than 40 kcal/mol. In some implementations, another filtering stage can use those vacuum dimers that do not sample the conformational space that is already sampled by liquid MD dimers. The latter is ensured by selecting those vacuum dimers that are conformationally far from the liquid dimers, that is the RMSD between vacuum MD dimers and liquid dimers is above a certain threshold.

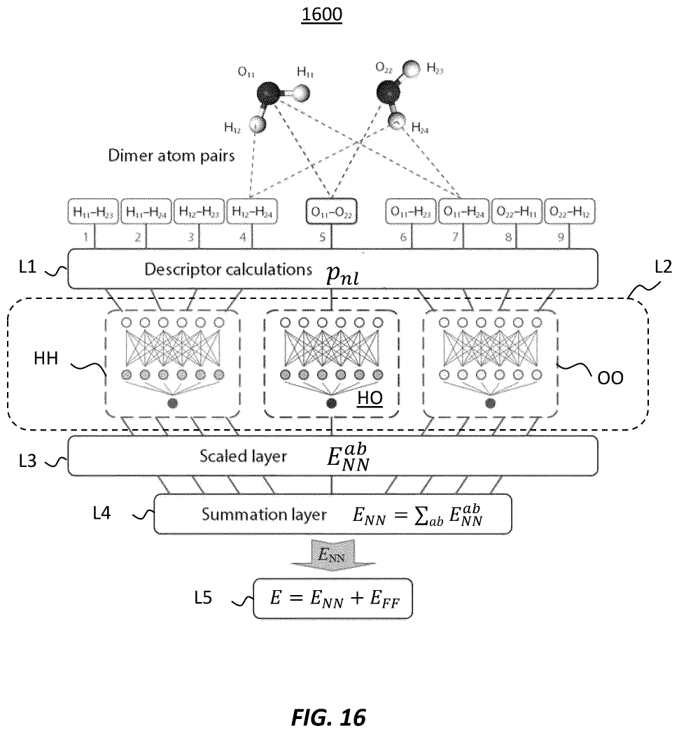

FIG. 16 shows a diagram of a neural network 1600 described by a CLM such as CLM 126 for pair interactions between two molecules (e.g., water molecules). The neural network 1600 may include trained neural network models configured to describe pair interaction fingerprints (e.g., H₁₁-H₂₃, as shown in FIG. 16). For example, the neural network models may include neural network model HH describing an interaction between two hydrogen atoms, neural network model HO describing an interaction between a hydrogen atom and an oxygen atom, and a neural network model OO describing an interaction between two oxygens atoms. It should be noted that for other molecular systems, other corresponding neural networks can be developed describing pairwise interactions between different atoms. To encode the pair interaction (e.g., pair H₁₁-H₂₃), atom pair symmetry functions (APSF) centered at the midpoint X between Hₙ and H₂₃ can be used. As shown in FIG. 16, a first layer L1 of the neural network model 1600 takes as input the information related to a pair of atoms (e.g., relative distance between the atoms in the pair as well as the locations of the atoms in the pairs of atoms and location of neighboring atoms) and calculates a descriptor pₙₗ for each pair of atoms, as described, for example, by equation 6 above. The descriptor pₙₗ for each pair of atoms is then input to a second layer L2 and into a corresponding neural network model (e.g., result of pₙₗ for H₁₁-H₂₃ are input into the neural network model HH). The output of a neural network model (e.g., output of neural network model HH) is then smoothly truncated (e.g., by a Tensorflow X-layer) to zero beyond a cutoff distance at the third layer L3 of the neural network 1600. The individual pair energy contributions are then summed to a final dimer energy correction ΔE_{NN} at a layer L4 of the neural network 1600, which is then added to the energy EFF output by the analytical Force Field to produce the total energy at layer L5 of the neural network 1600.

In various embodiments, the neural network models used for determining energy correction for the pair of atoms can be formulated for atomic types of the atoms in the pair of atoms. For example, a neural network model describing aromatic carbon-amide nitrogen may be used. Other neural network models may be developed to describe interactions of other atomic types, such as interactions of aliphatic carbon, carbonyl carbon, primary, secondary, ternary, or quaternary carbon, amine nitrogen, nitro nitrogen, pyridine nitrogen, quaternary nitrogen, carbonyl oxygen, hydroxyl oxygen, ether oxygen, acetal oxygen, ester oxygen, carboxylate oxygen, and/or the like with other atoms. This typification has several significant advantages. For example, having a neural network specific for atomic types, breaks-up the chemical interaction space into much smaller training subgroups so that model improvement is realized in segments and avoids pre-computing of all possible intermolecular interactions. For another example, having a neural network specific for atomic types also permits the resulting total hybrid accuracy of representation of the interaction to be high (e.g., on the order of convergence accuracy of the QM method chosen for the training set). Furthermore, as the amount of encoded information is limited, and because the fingerprint itself does not need to encode the chemical environment (‘type’) but can encode the relative orientation and bonded distortion of the interaction, having a neural network specific for atomic types allows for a smaller and therefore faster set of neural networks.

### Example Force Field Description

In various cases, polarizability in the force fields and nonadditive effects are important when determining two-body molecular interactions. Ability to respond to an electrical field is called a polarizational response. A polarizational response can make the force field more transferable, because molecules respond to an electrical field in similar manners, disregarding other small factors such as coupling of such response with other smaller effects such as dispersion interactions. Therefore, INM can include a two-body potential. For example, the underlining analytical force field may accurately determine effects of polarization on molecular interactions. In some cases, effects of polarization may include an anisotropic model of polarization in contrast to a simpler, less precise isotropic model of polarization.

In various cases, electrostatic forces may affect long-range molecular interactions and polarizability of molecules. Electrostatic forces can be the strongest of intermolecular forces at close, medium (2.5-5 Angstrom) and far distances (beyond 5 Angstrom). The presence of electrostatic forces in biomolecules is very prominent through water, polar, ion interactions, etc. Correct description of local electrostatic forces, thus, is important to reproduce non-additive energies. These non-additive energies may be caused by local charge distribution, which, in some cases, may lead to polarization self-consistent field “equilibration” of polarizable electronic clouds in molecules.

In various embodiments, electrostatic forces may be well described by long range algorithms like, for example, Particle-mesh Ewald (PME) algorithm. Truncation CLM and using analytical approach (e.g., MIM) for electrostatic treatment beyond e.g., a few Angstroms, such as, for example, 5 Angstrom may allow for fast molecular simulation (using algorithms such as PME, which scales similar to N log(N), where N is number of particles). Further, using MIM allows reducing the amount of dimer data that will be used for training CLMs (e.g., only molecules with distances less than a few Angstroms may be used for training CLMs).

In some implementations, the IHM may include the following features: The two-body non-bonded interactions may include electrostatic, exchange-repulsion and dispersion terms. The electrostatic and exchange-repulsion terms may be multipolar, and their radial dependence may be a Slater-like exponential so that the electrostatic and exchange-repulsion terms are better able to describe charge penetration and delocalization. Multibody effects can be modeled by anisotropic atomic polarizable dipoles interacting with the electrostatic term and with each other and iterated to self-consistent field (SCF) convergence on non-bonded step. In some implementations, there may be no explicit three-body terms, and may not include charge flux. The intermolecular parameters of MIM are determined by agreements with QM values of dimer and multimer energies, electrostatic potentials, and multipole moments of monomers. To aid transferability, the individual components to their corresponding QM counterparts may be matched, in addition to reproducing the total energy. The bonded interactions may be fitted to monomer QM energies computed at the df-MP2/aug-cc-pVTZ level of theory.

In various cases, the screening of the dispersion interaction in the intermediate and far range can be a many-body phenomenon and can be difficult to obtain from ab-initio calculations. Therefore, the close-range dispersion interaction may be obtained from a decomposition of dimer QM calculations, and then smoothly scaled down by a factor that is a property of the two interacting elements. The scaling factor may be specific to the periodic table membership and bond order, and the value is chosen to best reproduce densities of liquids. In some cases, for example, the values cutoff=4.5 Å and asymptotic reduction of 0.5 may be appropriate.

### Force Field Functional Form of an Example Molecular Interaction Model

A) MIM Charge Density function

In some implementations, for example, in the MIM calculations, the charge density of the electron cloud of atom ‘a’ located at position Rₐ can be written as:

$${ρ}_{a} ( r ) = {\hat{D}}_{a} \frac{{q}_{a}}{8 π {w}_{A}^{3}} exp ( - \vert r - {R}_{a} \vert / {w}_{A} ) , \qquad ( A1 )$$

where qₐ is the cloud charge and w_{A} is the MIM atom-type parameter that characterizes the cloud size. The capital letters ‘A’, ‘B’, and so on, may be used to denote the MIM atom-types respectively for atoms ‘a’, ‘b’, . . . ; similar notations are used for bond, bend and torsion atoms types (e.g. ‘AB’ denotes the MIM bond type for the pair of bonded atoms ‘a’ and ‘b’).

In some implementations, for example, the cloud charge may be written in the form:

$${q}_{a} = - {Z}_{A} + {\sum }_{b ∈ \{ a \}} {q}_{BA} \qquad ( A2 )$$

where Z_{A} is the charge of the “nucleus” for the atom type ‘A’, {a} denotes the set of atoms chemically bonded to atom ‘a’ and q_{BA} is the MTM bond charge transfer parameters (thus the equality q_{AB}=−q_{BA} providing for charge conservation).

In some implementations, for example, differential operator \hat{D}ₐ can be:

$${\hat{D}}_{a} ≡ 1 + {t}_{a} \frac{\partial }{\partial {R}_{a}} + \frac{1}{2} {( \frac{\partial }{\partial {R}_{a}} )}^{T} {ω}_{a} \frac{\partial }{\partial {R}_{a}} \qquad ( A3 )$$

where dimensionless vector tₐ and 3×3 dimensionless tensor ωₐ introduce p and d type density anisotropy, respectively. They can be related to the local atomic dipole Pₐ and atomic quadrupole Qₐ as:

Pₐ=qₐtₐ,

Qₐ=qₐωₐ (A4)

Vector tₐ is a sum of permanent and induced vectors:

tₐ=tₐ^{per}+tₐ^{ind} (A5)

In some implementations, for example, the induced part is written as

tₐ^{ind}=t_{A}^{max}τₐ (A6)

where t^{max}_{A} is the MIM atom-type parameter and τₐ is the dimensionless vector representing the MIM dynamic variable, its length being confined within interval (0, 1) by the restraint potential U_{IN}. The induced variable is iterated to convergence (1E-6 kcal/mol/A) of the electrostatic and exchange forces in an SCF (self-consistent-field) manner as is common in polarizable Force Fields. The extended Largrangian techniques may not be employed, instead the dipoles may be iterated explicitly to convergence.

In some implementations, for example, the permanent part in Eq. (A5) is written as:

$${t}_{a}^{per} = {\sum }_{b ∈ \{ a \}} {t}_{AB} {n}_{ab} \qquad ( A7 )$$

where n_{ab} is the unit vector, n_{ab}=R_{ab}/|R_{ab}| and R_{ab} is the vector directed from atom ‘a’ to neighboring atom ‘b’ and t_{AB} is the MIM bond-type parameter associated with permanent ‘chemical’ polarization or “charge shift” along the bond ‘ab’(in general t_{AB}≠t_{BA}). In some implementations, for example, the components of the local quadrupole tensor, can be calculated as:

$${( {ω}_{a} )}_{αβ} = 2 {\sum }_{bz ( a )} {Q}_{AB} ( {( {n}_{ab} )}_{α} {( {n}_{ab} )}_{β} - \frac{{δ}_{αβ}}{3} ) \qquad ( A8 )$$

where α,β=x, y, z and Q_{AB} are the MIM bond-type parameters. The atom “nucleus” (proportional to Dirac's delta-function) can be formally considered as the limiting case of Eq. (A1) replacing qₐ by Z_{A}, dropping operator \hat{D}ₐ and formally approaching zero by the width parameter.

B) Non-Valence Interactions

### Electrostatic Potential

In some implementations, for example, based on representation of Equation (A1) the potential φ_{ab} of electrostatic interaction of two-unit charge distributions ‘a’ and ‘b’ separated by a distance R_{ab}=|R_{b}−Rₐ| can be represented in the form:

$${φ}_{ab} ( {R}_{ab} , \text{?} , {t}_{b} ) = \text{?} {φ}_{ab}^{( 0 )} ( {R}_{ab} ) \qquad ( B1 )$$

$${φ}_{ab}^{( 0 )} ( r ) = \frac{1}{r} [ 1 - f ( {w}_{A} , {w}_{B} ) \text{?} - f ( {w}_{B} , {w}_{A} ) \text{?} ] \text{?}$$

$$f ( u , v ) ≡ \frac{{u}^{4} ( 3 {v}^{2} - {u}^{2} )}{{( {v}^{2} - {u}^{2} )}^{3}} + \frac{{u}^{3}}{2 ( {v}^{2} - \text{?}} r . \qquad ( B2 )$$

$$\text{?} \text{indicates text missing or illegible when filed}$$

Expanding equation (B1) one gets:

$${φ}_{ab} ( {R}_{ab} , {t}_{a} , {t}_{b} ) = {\sum }_{k = 0}^{4} {F}_{ab}^{( k )} ( {R}_{ab} , {t}_{a} , {t}_{b} ) {φ}_{ab}^{( k )} ( {R}_{ab} ) \qquad ( B3 )$$

where F's are the algebraic factors can be defined as:

$${F}_{ab}^{( 0 )} = 1 , \qquad ( B4 )$$

$${F}_{ab}^{( 1 )} = ( {t}_{b} {R}_{ab} ) - ( {t}_{a} {R}_{ab} ) - ( {t}_{a} {t}_{b} ) ,$$

$${F}_{ab}^{( 2 )} = - ( {t}_{a} R ) ( {t}_{b} R ) + \frac{1}{2} Sp {ω}_{a} {ω}_{b} + \frac{1}{2} ( {R}^{+} {ω}_{a} R ) + \frac{1}{2} ( {R}^{+} {ω}_{b} R ) + ( {R}^{+} {ω}_{a} {t}_{b} ) - ( {R}^{+} {ω}_{b} {t}_{a} ) ,$$

$${F}_{ab}^{( 3 )} = \frac{1}{2} ( {t}_{b} R ) ( {R}^{+} {ω}_{a} R ) - \frac{1}{2} ( {t}_{a} R ) ( {R}^{+} {ω}_{b} R ) + ( {R}^{+} {ω}_{a} {ω}_{b} R ) ,$$

$${F}_{ab}^{( 4 )} = \frac{1}{4} ( {R}^{+} {ω}_{a} R ) ( {R}^{+} {ω}_{b} R ) .$$

and potentials φ^{{k}}_{ab} satisfy the recurrence relations:

$${φ}_{ab}^{( k + 1 )} ( R ) = \frac{1}{R} \frac{d}{dR} {φ}_{ab}^{( k )} ( R ) , k = 0 , 1 , ... \qquad ( B5 )$$

The ES potential U^{ES} between the two clouds can be written as:

U_{ab}^{ES}(R_{ab})=332.064·qₐq_{b}φ_{ab}(R_{ab})

The potentials are in kcal/mol, the components of vectors R_{ab} and t are in Å, charges are in a.u. This formula is also valid for “nucleus”-cloud and “nucleus”-“nucleus” interactions by replacing the cloud charges with the nucleus charges, nullifying vectors t and tensor o for the core, with the corresponding width parameter(s) formally approaching zero.

### Exchange Potential

In some implementations, for example, the multipolar EX potential UEX can be written similarly to that of ES:

U_{ab}^{EX}=332.064·C_{A}^{EX}C_{B}^{EX}χ_{ab}(R_{ab}) (B6)

where C^{EX}_{A}, C^{EX}_{B} are the force parameters for atom types ‘A’ and ‘B’, and

$${χ}_{ab} ( {R}_{ab} , {t}_{a} , {t}_{b} ) = {\sum }_{k = 0}^{4} {F}_{ab}^{( k )} ( {R}_{ab} , {t}_{a} , {t}_{b} ) {χ}_{ab}^{( k )} ( {R}_{ab} ) \qquad ( B7 )$$

The algebraic factors F were defined in Eq. (B4), potentials χ^{{k}}_{ab}(R_{ab}) satisfy the recurrence relations in Eq. (B5)

$${χ}_{ab}^{( 0 )} ( r ) ≡ {u}_{A} {u}_{B} \frac{{e}^{- r / {u}_{B}} - {e}^{- r / {u}_{A}}}{( {u}_{B} - {u}_{A} ) r} \qquad ( B8 )$$

where u_{A} and u_{B} are the EX width parameters that generally differ from ES parameters w_{A} and w_{B}. Φ_{ab}⁽⁰⁾ represents a functional form of the exchange potential.

### Dispersion Potential

U_{ab}^{DS}=−332.064·(C_{A}^{DS6}C_{B}^{DS6}ψ₆(R_{ab})+C_{A}^{DS8}C_{B}^{DSS}ψ₈(R_{ab})) (B9)

where C^{DS6}, C^{DS8} are the force constants and ψ's are the Tang-Toennies's functions:

$$\text{?} ( r ) = \text{?} [ 1 - \text{?} {\sum }_{k = 1}^{n} \frac{1}{k !} {( \frac{2 r}{{v}_{A} + {v}_{B}} )}^{k} ] \qquad ( B10 )$$

$$\text{?} \text{indicates text missing or illegible when filed}$$

v_{A} and v_{B} being the dispersion width parameters that generally differ from those for electrostatics and exchange.

### Induction Potential

In some implementations, for example, the anharmonic restraint potential for the atom ‘a’ can be represented as:

$${U}_{a}^{IN} ( {τ}_{a} ) = 2240.88 \text{?} \frac{{( {q}_{a} {t}_{A}^{max} )}^{2}}{{α}_{A} {M}_{a}} \frac{\text{?} + \text{?} {{s}_{AB} ( {τ}_{a} {n}_{ab} )}^{2}}{1 + \sqrt{1 - {( {τ}_{a} )}^{2}}} , \text{?} = 1 + \frac{1}{3} \text{?} {s}_{AB} \qquad ( B11 )$$

$$\text{?} \text{indicates text missing or illegible when filed}$$

where α_{A} is the MIM parameter characterizing polarizability prescribed to atom-type A while bond-type parameters s_{AB} describe the anisotropy of the restraint potential (generally s_{AB}≠S_{BA}), the vector τₐ was introduced in Eq. (A6). Note that the force due to potential in Eq. (B11) approaches infinity as τₐ approaches 1. This peculiarity of the MIM restraint potential avoids the polarization catastrophe and provides the existence of a solution of the problem of improvement of electron density in any physically reasonable external field.

C) Induced Dipoles Implementation

It can be seen from equations (B1-B11) that the system depends on polarizable part of clouds tₐ^{ind}=t_{A}^{max}τₐ that responds to electrostatic fields of other cloud charges, “nuclei”, cloud dipoles, cloud quadrupoles as well as other polarizable clouds in the system. These equations of self-consistent polarizable clouds can be represented as typical quadratic form of induced polarizable shifts or dipoles E=E_{τ-independen}+6Στₐτ_{b}+ΣCₐτₐ+ΣC_{b}τ_{b}. This quadratic form is optimized and/or improved with steepest descent with respect to polarizable clouds vector τₐ at the steps of molecular dynamics until it reaches a small force threshold, which can be, for example, about 1E-6 kcal/mol/A.

D) Valence Interactions

In some implementations, for example, similar to the MMFF-94 force field, the Hamiltonian of valence interaction can be given in the form:

H=H^{BS}+H^{AB}+H^{StBn}+H^{OOP}+H^{TORS} (C1)

where the terms correspond respectively to bond stretching (BS), angle bending (AB), stretch-bend (StBn), out-of-plane bending (OOP), and torsion interactions (TORS).

In some implementations, for example, the bond stretching component is of the form:

$${H}^{BS} = \frac{1}{2} \sum {k}_{IJ}^{( b )} Δ {{r}_{ij}}^{2} ( 1 + {c}_{b} Δ {r}_{ij} + \frac{7}{12} {{c}_{b}}^{2} Δ {{r}_{ij}}^{2} ) \qquad ( C2 )$$

$$where$$

$$Δ {r}_{ij} = \vert {r}_{i} - {r}_{j} \vert - {r}_{IJ}^{( 0 )} \qquad ( C3 )$$

In Eq. (C2), the sum is over the bond pairs, I and J are the indices of the atomic types ascribed respectively to atoms i and j, r_{I,J}⁽⁰⁾ and k_{I,J}^{(b)} are respectively the equilibrium (or reference) bond length and the bond force constant determined by the atomic type pair and the specific bond type index, and c_{b} is a model parameter.

In some implementations, for example, the angle bending term is given by:

$${H}^{AB} = \frac{1}{2} \sum {k}_{IJK}^{( a )} {{Δφ}_{ijk}}^{2} ( 1 + {c}_{a} {Δφ}_{ijk} ) \qquad ( C4 )$$

where the summation is performed over the bend triplets i-j, j-k, the indices I, J and K corresponding to the atomic types ascribed respectively to atoms i, j and k. Other notations are:

Δφᵢⱼₖ=φᵢⱼₖ−φ_{IJK}⁽⁰⁾ (C5)

where φijk is the angle between i-j and k-j bonds, φIJK{0} and kIJK{a} are the equilibrium (or reference) angle and the force constant determined by the atomic type triplet and the specific angle type index, and ca is a model parameter.

In some implementations, for example, the stretch-bend interaction is given by:

H^{St-Bn}=ΣΔφᵢⱼₖ(k_{IJK}^{(ba)}Δrᵢⱼ+k_{KJI}^{(ba)}Δrₖⱼ) (C6)

where the summation is similar to Eq. (C4). The bond length and angle deviations, Δrᵢⱼ, Δrₖⱼ and Δφᵢⱼₖ are defined by Eqs. (C3) and (C5), k^{(ba)}_{IJK} and k^{(ba)}_{KJI} are the force constants for the interaction of ij and kj bond-angle, respectively. These constants are determined by the atomic type triplet and the specific stretch-bend type index.

In some implementations, for example, the out-of-plane interaction is given by:

$${H}^{OOP} = \frac{1}{2} \sum {k}_{IJKL}^{( oop )} ( {{Δχ}_{ikl}}^{2} + {{Δχ}_{kli}}^{2} + {{Δχ}_{lik}}^{2} ) \qquad ( C7 )$$

where the sum is over bond tetrahedrals i-j-k-1 with the 3-bond central (or vertex) atom j and the bonds i-j, k-j, l-j, the indices I, J, K and L correspond to the atomic types ascribed respectively to atoms i, j, k, and l. The angle deviation Δχᵢₖₗ is defined as the angle between the vector (rᵢ-rⱼ) and the plane defined by vectors (rₖ-rₗ) and (rₗ-rⱼ); that is

$$sin ( {Δχ}_{ikl} ) = \frac{{n}_{ij} \cdot ( {n}_{kj} \times {n}_{lj} )}{\vert {n}_{kj} \times {n}_{lj} \vert} \qquad ( C8 )$$

$$where$$

$${n}_{aj} = ( {r}_{a} - {r}_{j} ) / \vert {r}_{a} - {r}_{j} \vert , a = i , k , l \qquad ( C9 )$$

In some implementations, for example, the force constant k_{IJKL} is determined by the atomic type quartet IJKL. In some implementations, for example, the torsion component is of the form

$${H}^{rORS} = \frac{1}{2} \sum [ {V}_{IJKL}^{( 1 )} ( 1 + cos ϕ ) + {V}_{IJKL}^{( 2 )} ( 1 - cos 2 ϕ ) + {V}_{IJKL}^{( 3 )} ( 1 + cos 3 ϕ ) ] \qquad ( C10 )$$

where the sum is over bond quartets i-j, j-k, k-l, the indices I, J, K, and L correspond to the atomic types ascribed respectively to atoms i, j, k, and l. ø is a standard torsion angle defined as the angle between the planes i-j-k and j-k-l, given by:

$$cos ϕ = {m}_{ij} \cdot {m}_{ik} \qquad ( C11 )$$

$$where$$

$${m}_{ij} = \frac{{n}_{ij} - {n}_{jk} ( {n}_{jk} \cdot {n}_{ij} )}{\sqrt{1 - {( {n}_{ij} \cdot {n}_{jk} )}^{2}}} , {m}_{kl} = \frac{{n}_{kl} - {n}_{jk} ( {n}_{jk} \cdot {n}_{kl} )}{\sqrt{1 - {( {n}_{kl} \cdot {n}_{jk} )}^{2}}} \qquad ( C12 )$$

In some implementations, for example, the force constants V_{IJKL} are determined by the atomic type quartet and two specific indices, the first relating to the type of the central bond j-k, while the second one relates to the hybridization state of atoms j and k.

It should be appreciated that the above detailed embodiments of the present disclosure are only to exemplify or explain principles of the present disclosure and not to limit the present disclosure. Therefore, any modifications, equivalent alternatives and improvement, etc. without departing from the scope of the present disclosure shall be included in the scope of protection of the present disclosure. Meanwhile, appended claims of the present disclosure aim to cover all the variations and modifications falling under the scope and boundary of the claims or equivalents of the scope and boundary.

The above-described embodiments can be implemented in any of numerous ways. For example, embodiments may be implemented using hardware, software or a combination thereof. When implemented in software, the software code can be stored (e.g., on non-transitory memory) and executed on any suitable processor or collection of processors, whether provided in a single computer or distributed among multiple computers.

Further, it should be appreciated that a compute device including a computer can be embodied in any of a number of forms, such as a rack-mounted computer, a desktop computer, a laptop computer, netbook computer, or a tablet computer. Additionally, a computer can be embedded in a device not generally regarded as a computer but with suitable processing capabilities, including a smartphone, smart device, or any other suitable portable or fixed electronic device.

Also, a computer can have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer can receive input information through speech recognition or in other audible format.

Such computers can be interconnected by one or more networks in any suitable form, including a local area network or a wide area network, such as an enterprise network, and intelligent network (IN) or the Internet. Such networks can be based on any suitable technology and can operate according to any suitable protocol and can include wireless networks, wired networks or fiber optic networks.

The various methods or processes outlined herein can be coded as software that is executable on one or more processors that employ any one of a variety of operating systems or platforms. Additionally, such software can be written using any of a number of suitable programming languages and/or programming or scripting tools, and also can be compiled as executable machine language code or intermediate code that is executed on a framework or virtual machine.

In this respect, various disclosed concepts can be embodied as a computer-readable storage medium (or multiple computer-readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the disclosure discussed above. The computer-readable medium or media can be transportable, such that the program or programs stored thereon can be loaded onto one or more different computers or other processors to implement various aspects of the present disclosure as discussed above.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

The terms “program” or “software” or “software stack” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but can be distributed in a modular fashion amongst a number of different computers or processors to implement various aspects of the disclosure.

Computer-executable instructions can be in many forms, such as program modules, executed by one or more computers or other devices. Generally, program modules include routines, programs, objects, components, data structures, etc. that perform particular tasks or implement particular abstract data types. Typically the functionality of the program modules can be combined or distributed as desired in various embodiments.

Also, various concepts can be embodied as one or more methods, of which an example has been provided. The acts performed as part of the method may be ordered in any suitable way. Accordingly, embodiments can be constructed in which acts are performed in an order different than illustrated, which can include performing some acts simultaneously, even though shown as sequential acts in illustrative embodiments. All publications, patent applications, patents, and other references mentioned herein are incorporated by reference in their entirety.

1.-62. (canceled)

63. A method, comprising:
determining, at a processor, a first plurality of atomic interactions between a first set of atoms and a second set of atoms using a Newtonian-based molecular interaction model (EPh), the EPh including responses to an electric field external to at least one of the first set of atoms or the second set of atoms, an inductive accounting of many-body non-additive interactions, and a representation of electrostatic interactions and electrostatic response interactions;
providing a plurality of inputs to a plurality of neural network models (dENN) to determine a second plurality of atomic interactions between the first set of atoms and the second set of atoms, each input from the plurality of inputs being a representation of at least one atom of the first set of atoms or the second set of atoms;
training the dENN by reducing an error between (1) an interaction produced by a combination of the first plurality of atomic interactions and the second plurality of atomic interactions and (2) an interaction between the first set of atoms and the second set of atoms computed using methods that are based on quantum mechanics and that are distinct from the EPh;
modifying the EPh by reducing an error between (1) the interaction produced by the combination of the first plurality of atomic interactions and the second plurality of atomic interactions and (2) the interaction between the first set of atoms and the second set of atoms computed using the methods that are based on quantum mechanics;
receiving a representation of a third set of atoms;
inputting the representation of the third set of atoms into the EPh and the dENN to output a prediction associated with an energy or a state of the third set of atoms; and
sending a signal to a user device to present the prediction.

64. The method of claim 63, wherein the prediction includes at least one of a partition function, free energy, enthalpy, entropy, density, hydration free energy, conductivity, heat of vaporization, diffusion properties, binding free energy, proportion of reactants, or non-equilibrium properties.

65. The method of claim 63, wherein the combination is realized by at least one of:
adding the first plurality of atomic interactions and the second plurality of atomic interactions;
using a substitution of at least one parameter of the EPh by an output neural network model from the dENN; or
applying the first plurality of atomic interactions to a first subset of the third set of atoms and by applying the second plurality of atomic interactions to a second subset of the third set of atoms.

66. The method of claim 65, further comprising:
smoothly, over a predefined interval and using a neural network analytical layer, switching an output of the plurality of neural network models to zero at a predefined distance between the first set of atoms and the second set of atoms.

67. The method of claim 63, wherein the first set of atoms includes only a first single atom and the second set of atoms includes only a second single atom.

68. The method of claim 63, wherein the first set of atoms includes a first atom and at least one neighbor atom of the first atom and the second set of atoms includes a second atom and at least one neighbor atom of the second atom.

69. The method of claim 63, wherein the first set of atoms and the second set of atoms are within at least a portion of a molecule common to the first set of atoms and the second set of atoms.

70. The method of claim 63, wherein the first set of atoms is the same as the second set of atoms.

71. The method of claim 63, wherein the first set of atoms includes atoms within a first molecule and the second set of atoms includes atoms within a second molecule different from the first molecule.

72. The method of claim 63, wherein a neighborhood is defined by a predetermined distance from a selected atom, two atoms, or from a geometrically assigned interaction center, or a predetermined number of bonds away from the selected atom or atoms.

73. The method of claim 72, wherein a geometrically assigned interaction center is a reference point and is located at a center of a line connecting a first atom selected from the first set of atoms and a second atom selected from the second set of atoms.

74. The method of claim 63, wherein the representation of the at least one of the first set of atoms or the second set of atoms includes at least one of an orientation of an atom from the first set of atoms or the second set of atoms, a neighborhood of an atom from the first set of atoms or the second set of atoms, a type of an atom first set of atoms or the second set of atoms, or a relative location of an atom from first set of atoms or the second set of atoms.

75. The method of claim 63, wherein the representation of the at least one of the first set of atoms or the second set of atoms has a predetermined length not dependent on a rotation, a translation, or a reordering of member atoms in the at least one of the first set of atoms or the second set of atoms, or reordering of the sets themselves.

76. The method of claim 63, wherein each neural network from the plurality of neural network models is configured to describe either a distinct atom type or a distinct atomic pair type.

77. The method of claim 63, wherein a type of an atom for an atom from the first set of atoms or from the second set of atoms is determined based on at least one of a membership of the atom in the periodic table, or the atoms in a neighborhood of the atom.

78. The method of claim 63, wherein an atomic pair type for an atomic pair having the first atom from the first set of atoms and the second atom from the second set of atoms is determined based on at least one of a membership of the first and the second atoms in the periodic table, or the atoms in a neighborhood of the atomic pair.

79. The method of claim 76, wherein the distinct atom type includes an aromatic carbon, an aliphatic carbon, a carbonyl oxygen, an ether oxygen, or a hydroxyl oxygen.

80. The method of claim 63, wherein the interaction between the first set of atoms and the second set of atoms is an overall interaction computed using quantum mechanical methods, the overall interaction between the first set of atoms and the second set of atoms is determined based on at least one of: one or more interactions of a single molecule, pairs of molecules, triples of molecules, a molecule and an ion, a molecule and a charge, a molecule, or an applied electric field.

81. The method of claim 63, wherein the interaction between the first set of atoms and the second set of atoms is an overall interaction computed using quantum mechanical methods, the overall interaction between the first set of atoms and the second set of atoms is determined based on energy subcomponents, the energy subcomponents including at least one of: dispersion, exchange-repulsion, electrostatic, or induction interactions.

82. The method of claim 63, wherein the interaction between the first set of atoms and the second set of atoms is determined based on energy subcomponents, the energy subcomponents include at least one of: nuclear quantum effects, polarization induced by fields external to at least one of the first set of atoms or the second set of atoms, or polarization induced by a molecule.

83. The method of claim 63, wherein the electrostatic response interactions include at least one of induction, polarization or charge transfer.

84. The method of claim 63, wherein a set of inputs to EPh includes at least one of monopole, dipole, quadrupole, or octupole, having a radial accuracy within fifteen percent.

85. The method of claim 63, wherein modifying the EPh includes adjusting parameters of the EPh such that EPh is configured to generate an output that reproduces experimentally observable or measurable macroscopic properties of collections of molecules.

86. The method of claim 63, wherein modifying the EPh includes adjusting parameters of the EPh such that an error between an output of the EPh and an output computed by quantum mechanical methods is reduced.

87. The method of claim 86, wherein the output includes at least one of: energies of interaction between molecules, partial charges, induced charge distribution changes, or correlated charge fluctuation responses.

88. The method of claim 63, further comprising:
determining an induction response by evaluating a polarization for the first and the second set of atoms, wherein the evaluating includes computing the interaction produced by a combination of the first plurality of atomic interactions and the second plurality of atomic interactions placed within the electric field.

89. The method of claim 88, wherein the electric field is caused by a charged particle placed at a location within a proximity threshold to the first and the second set of atoms.

90. The method of claim 63, further comprising truncating the molecular interaction model (EPh) at a long range.

91. A method, comprising:
determining, at a processor, a first plurality of atomic interactions between a first set of atoms and a second set of atoms using a Newtonian-based analytical molecular interaction model (EPh);
providing a plurality of inputs to a plurality of neural network models (dENN) to determine a second plurality of atomic interactions between the first set of atoms and the second set of atoms, each input from the plurality of inputs being a representation of at least one atom of the first set of atoms or the second set of atoms;
training the dENN by reducing an error between (1) an interaction produced by a combination of the first plurality of atomic interactions and the second plurality of atomic interactions and (2) an interaction between the first set of atoms and the second set of atoms computed using methods that are based on quantum mechanics and that are distinct from the EPh;
receiving a representation of a third set of atoms;
inputting the representation of the third set of atoms into the EPh and the dENN to output a prediction associated with an energy or a state of the third set of atoms; and
sending a signal to a user device to present the prediction.

92. The method of claim 91, wherein the EPh includes a representation of electromagnetic interactions, the electromagnetic interactions including at least one of electrostatic interactions, induction, polarization or charge transfer.
